SEQUENCE LISTING (300 sequences)

SEQ ID NO: 1 - DNA, 1338 bp, synthetic construct
  misc_feature 1..1338: note = Synthetic Polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagttatggtgcttttgactactggggccagggaaccctggtcacagtctcgagcgcctccaccaagggcccatcggtcttccccctggcaccctcctccaagagcacctctgggggcacagcggccctgggctgcctggtcaaggactacttccccgaaccggtgacagtctcgtggaactcaggagccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatctcgggaggagatgaccaagaaccaggtcagcctgacttgcctggtcaaaggcttctatcccagcgacatcgccgtggagtgggagagcaacgggcagccggagaacaactacaagaccacgcctcccgtgctggactccgacggctccttcttcctctatagcaagctcaccgtggacaagtccaggtggcagcaggggaacgtcttctcatgctccgtgatgcatgaggctctgcacaaccactacacgcagaagagcctctccctgtctccgggttaa

SEQ ID NO: 2 - protein, 445 aa, synthetic construct
  REGION 1..445: note = Synthetic Polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3-54 - skipped (52 entries)

SEQ ID NO: 55 - DNA, 648 bp, synthetic construct
  misc_feature 1..648: note = Synthetic Polynucleotide
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcagatgcacccgcgcgccccgaagaccttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 56 - protein, 215 aa, synthetic construct
  REGION 1..215: note = Synthetic Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57-106 - skipped (50 entries)

SEQ ID NO: 107 - DNA, 678 bp, synthetic construct
  misc_feature 1..678: note = Synthetic Polynucleotide
  source 1..678: mol_type = other DNA
cagcctgtgctgactcagccggcttccctctctgcatctcctggggcatcagccagtctcacctgcaccttgcgcagtggcatcaacgttagagattacaggatattctggtaccagcagaagccagggagtcctccccagtatctcctgaggtacaaaagcgcatcagataagcagcagggctctggagtccccagccgcttctctgggtccaaagatgcttcggccaatgcagggattttactcatctctgggctccagtctgaggatgaggctgactattactgtatgatttggcaccacgattcggaggggcatgcttttgtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 108 - protein, 225 aa, synthetic construct
  REGION 1..225: note = Synthetic Polypeptide
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - DNA, 648 bp, synthetic construct
  misc_feature 1..648: note = Synthetic Polynucleotide
  source 1..648: mol_type = other DNA
tcctatgtgctgactcagccaccctcagtgtcagtggccccaggaaagacggccaggattacctgtgggggaaacaaaattggacaccgcgccgtgcactggtaccagcagaagccaggccaggcccctgtgctggtcatctattatacctatgagcggccctcagggattcctgagcgattctctggctccaactctgggaacacggccaccctgaccatcagcagggtcgaagccggggatgaggccgactattactgtcaggtgtgggattggtacagcgaggggggggttgtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 110 - skipped

SEQ ID NO: 111 - DNA, 651 bp, synthetic construct
  misc_feature 1..651: note = Synthetic Polynucleotide
  source 1..651: mol_type = other DNA
cagtctgtgttgacgcagccgccctcagtgtctgcggccccaggacagaaggtcaccatctcctgctctggaagcagctccaatattgagactggttctgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatgacagcctgcctggatgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 112 - protein, 216 aa, synthetic construct
  REGION 1..216: note = Synthetic Polypeptide
QSVLTQPPSVSAAPGQKVTISCSGSSSNIETGSVSWYQQLPGTAPKLLIYDNNKRPSGIPDRFSGSKSGTSATLGITGLQTGDEADYYCGTWDDSLPGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - DNA, 348 bp, synthetic construct
  misc_feature 1..348: note = Synthetic Polynucleotide
  source 1..348: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagttatggtgcttttgactactggggccagggaaccctggtcacagtctcgagc

SEQ ID NO: 114 - protein, 116 aa, synthetic construct
  REGION 1..116: note = Synthetic Polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 115-166 - skipped (52 entries)

SEQ ID NO: 167 - DNA, 324 bp, synthetic construct
  misc_feature 1..324: note = Synthetic Polynucleotide
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcagatgcacccgcgcgccccgaagaccttcggccaagggaccaaggtggaaatcaaa

SEQ ID NO: 168 - protein, 108 aa, synthetic construct
  REGION 1..108: note = Synthetic Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIK

SEQ ID NO: 169-204 - skipped (36 entries)

SEQ ID NO: 205 - DNA, 330 bp, synthetic construct
  misc_feature 1..330: note = Synthetic Polynucleotide
  source 1..330: mol_type = other DNA
cagtctgtgttgacgcagccgccctcagtgtctgcggccccaggacagaaggtcaccatctcctgctctggaagcagctccaatattgagactggttctgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatgacagcctgcctggatgggtgttcggcggagggaccaagctgaccgtccta

SEQ ID NO: 206 - protein, 110 aa, synthetic construct
  REGION 1..110: note = Synthetic Polypeptide
QSVLTQPPSVSAAPGQKVTISCSGSSSNIETGSVSWYQQLPGTAPKLLIYDNNKRPSGIPDRFSGSKSGTSATLGITGLQTGDEADYYCGTWDDSLPGWVFGGGTKLTVL

SEQ ID NO: 207 - DNA, 657 bp, synthetic construct
  misc_feature 1..657: note = Synthetic Polynucleotide
  source 1..657: mol_type = other DNA
gaaatagtgatgacgcagtctccagccaccctgtctgtgtctccaggggaaagagccaccctctcctgcagggccagtcagacggttaagaataatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataacaactggttgcccatcaacccctataccttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 208 - protein, 218 aa, synthetic construct
  REGION 1..218: note = Synthetic Polypeptide
EIVMTQSPATLSVSPGERATLSCRASQTVKNNLAWYQQKPGQAPRLLIYGASTRATGIPARFSGSGSGTEFTLTISSLQSEDFAVYYCQQYNNWLPINPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - DNA, 333 bp, synthetic construct
  misc_feature 1..333: note = Synthetic Polynucleotide
  source 1..333: mol_type = other DNA
gaaatagtgatgacgcagtctccagccaccctgtctgtgtctccaggggaaagagccaccctctcctgcagggccagtcagacggttaagaataatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataacaactggttgcccatcaacccctataccttcggccaagggaccaaggtggaaatcaaa

SEQ ID NO: 210 - protein, 111 aa, synthetic construct
  REGION 1..111: note = Synthetic Polypeptide
EIVMTQSPATLSVSPGERATLSCRASQTVKNNLAWYQQKPGQAPRLLIYGASTRATGIPARFSGSGSGTEFTLTISSLQSEDFAVYYCQQYNNWLPINPYTFGQGTKVEIK

SEQ ID NO: 211-220 - skipped (10 entries)

SEQ ID NO: 221 - DNA, 357 bp, synthetic construct
  misc_feature 1..357: note = Synthetic Polynucleotide
  source 1..357: mol_type = other DNA
cagcctgtgctgactcagccggcttccctctctgcatctcctggggcatcagccagtctcacctgcaccttgcgcagtggcatcaacgttagagattacaggatattctggtaccagcagaagccagggagtcctccccagtatctcctgaggtacaaaagcgcatcagataagcagcagggctctggagtccccagccgcttctctgggtccaaagatgcttcggccaatgcagggattttactcatctctgggctccagtctgaggatgaggctgactattactgtatgatttggcaccacgattcggaggggcatgcttttgtgttcggcggagggaccaagctgaccgtccta

SEQ ID NO: 222 - protein, 119 aa, synthetic construct
  REGION 1..119: note = Synthetic Polypeptide
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVL

SEQ ID NO: 223-224 - skipped (2 entries)

SEQ ID NO: 225 - protein, 8 aa, synthetic construct
  REGION 1..8: note = Synthetic Polypeptide
GFTFSSYA

SEQ ID NO: 226 - protein, 8 aa, synthetic construct
  REGION 1..8: note = Synthetic Polypeptide
ISGSGGST

SEQ ID NO: 227 - protein, 9 aa, synthetic construct
  REGION 1..9: note = Synthetic Polypeptide
AKSYGAFDY

SEQ ID NO: 228-239 - skipped (12 entries)

SEQ ID NO: 240 - protein, 6 aa, synthetic construct
  REGION 1..6: note = Synthetic Polypeptide
QSISSY

SEQ ID NO: 241-253 - skipped (13 entries)

SEQ ID NO: 254 - protein, 10 aa, synthetic construct
  REGION 1..10: note = Synthetic Polypeptide
QQMHPRAPKT

SEQ ID NO: 255-285 - skipped (31 entries)

SEQ ID NO: 286 - protein, 9 aa, synthetic construct
  REGION 1..9: note = Synthetic Polypeptide
SGINVRDYR

SEQ ID NO: 287-291 - skipped (5 entries)

SEQ ID NO: 292 - protein, 7 aa, synthetic construct
  REGION 1..7: note = Synthetic Polypeptide
YKSASDK

SEQ ID NO: 293-297 - skipped (5 entries)

SEQ ID NO: 298 - protein, 13 aa, synthetic construct
  REGION 1..13: note = Synthetic Polypeptide
MIWHHDSEGHAFV

SEQ ID NO: 299-300 - skipped (2 entries)

(19) United States
(12) Patent Application Publication
(10) Pub. No.: US 2025/0171537 A1
(43) Pub. Date: May 29, 2025

(54) METHOD OF TREATING DISEASE USING ANTI-CD47 X ANTI-MESOTHELIN ANTIBODIES AS A SOLE AGENT AND IN COMBINATION WITH ANTI-PD-1 ANTIBODIES

(71) Applicant: Novimmune SA, Geneva (CH)

(72) Inventors: Jose Maria SARO SUAREZ, Geneva (CH); Walter G. FERLIN, Collonges-Sous-Salève (FR)

(21) Appl. No.: 18/961,667

(22) Filed: Nov. 27, 2024

Related U.S. Application Data
(60) Provisional application No. 63/603,418, filed on Nov. 28, 2023.

(51) Int. Cl. C07K 16/28; A61K 39/00; C07K 16/30

(52) U.S. Cl. CPC C07K 16/2803; C07K 16/2818; C07K 16/30; A61K 2039/505; A61K 2039/54; A61K 2039/545; C07K 2317/31; C07K 2317/33; C07K 2317/92; C07K 2317/94

(57) ABSTRACT

The invention relates method of treatment of cancer using novel bispecific antibodies carrying a different specificity for each binding site of the immunoglobulin molecule, where one of the binding sites is specific for CD47 and the second is specific for mesothelin (MSLN). In addition, the disclosure contains methods of treatment using novel bispecific antibodies in combination with anti-PD-1 antibody.

### RELATED APPLICATIONS

This application claims priority to and the benefit of U.S. Provisional Application No. 63/603,418, filed on Nov. 28, 2023. The contents of which are incorporated herein by reference in its entirety.

### REFERENCE TO AN ELECTRONIC SEQUENCE LISTING

The contents of the electronic sequence listing (NOVI_059_001US_SeqList_ST26.xml; Size: 99,348 bytes; and Date of Creation: Nov. 25, 2024) are herein incorporated by reference in its entirety.

### BACKGROUND

CD47 or Integrin-Associated-Protein (IAP) is a ubiquitous 50 kDa transmembrane glycoprotein with multiple functions in cell-cell communication. It interacts with multiple ligands, such as integrins, SIRPα (Signal Regulatory Protein alpha), SIRPγ and thrombospondins (Oldenborg, P. A., CD47: A Cell Surface Glycoprotein Which Regulates Multiple Functions of Hematopoietic Cells in Health and Disease, ISRN Hematol. 2013; 2013:614619; Soto-Pantoja D R, et al., Therapeutic opportunities for targeting the ubiquitous cell surface receptor CD47 (2012), Expert Opin Ther Targets. 2013 January; 17(1):89-103; Sick E, et al., CD47 Update: a multifaced actor in the tumor microenvironment of potential therapeutic interest, Br J Pharmacol. 2012 December; 167(7):1415-30).

The widespread expression of CD47 in healthy tissues brings the question of treatment safety and efficacy: first, targeting CD47 with a neutralizing monoclonal antibody (Mab) could affect healthy cells, resulting in severe toxicities as shown in preclinical studies with mice and cynomolgus monkeys (Willingham S B, et al., Proc Natl Acad Sci USA. 2012 Apr. 24; 109(17):6662-7; Weiskopf K, et al., Engineered SIRPα Variants as Immunotherapeutic Adjuvants to Anticancer Antibodies, Science. 2013 Jul. 5; 341(6141):88-91). Second, even if severe toxicities could be avoided or mitigated by using alternative formats (Weiskopf K, et al., Science. 2013 Jul. 5; 341(6141):88-91), broad expression of CD47 could still cause a rapid elimination of CD47-binding molecules through target-mediated drug disposition resulting in poor pharmacokinetics and decreased efficacy.

Accordingly, there exists a need for antibodies and therapeutics that enable targeting of CD47 and overcome these obstacles. Furthermore, there exists a need to determine the therapeutically effective dose and schedule of particular anti-CD47 targeting antibodies and their potential use as a combination therapy with other tumor associated antigen targeting agents. Moreover, there exists a need to develop therapies that can treat cancers that are resistant to the standard of care treatment alone.

### SUMMARY

The present disclosure relates to the therapeutic methods, uses, and compositions comprising bispecific antibodies that recognize CD47 and mesothelin. The present disclosure relates to the bispecific antibody is Ka3×O38 or a fragment thereof and the clinical use thereof. Ka3×O38 refers to “NI-1801”.

The disclosure also provides bispecific antibodies that include at least a first arm that is specific for CD47. In some embodiments, the first arm is specific for at least human CD47. In some embodiments, the first arm recognizes human CD47 and is also cross-reactive for at least one other non-human CD47 protein, such as, by way of non-limiting example, non-human primate CD47, e.g., cynomolgus monkey CD47, and/or rodent CD47. In some embodiments, these anti-CD47 monoclonal antibodies inhibit the interaction between CD47 and signal-regulatory protein alpha (SIRPa). In some embodiments, these bispecific antibodies inhibit the interaction between human CD47 and human SIRPa. The invention also includes antibodies that bind to the same epitope as a bispecific antibody disclosed herein and inhibits the interaction between CD47 and SIRPα, e.g., between human CD47 and human SIRPα.

The disclosure also provides bispecific antibodies that recognize CD47 and a second target. The disclosure allows for the identification, production and purification of bispecific antibodies that are undistinguishable in sequence from standard antibodies and where one of the binding sites is specific for CD47 and the second binding site is specific for another target, for example a tumor-associated antigen (TAA). In some embodiments, the TAA is an antigen expressed on a cancer cell's cell surface. In some embodiments, the cancer cell is selected from a lung cancer cell, a bronchial cancer cell, a prostate cancer cell, a breast cancer cell, a colorectal cancer cell, a pancreatic cancer cell, an ovarian cell, a leukemia cancer cell, a lymphoma cancer cell, an esophageal cancer cell, a liver cancer cell, a urinary and/or bladder cancer cell, a renal cancer cell, an oral cavity cancer cell, a pharyngeal cancer cell, a uterine cancer cell, and/or a melanoma cancer cell.

The bispecific antibodies of the disclosure that bind at least CD47 and fragments thereof serve to modulate, block, inhibit, reduce, antagonize, neutralize or otherwise interfere with the functional activity of CD47. Functional activities of CD47 include, by way of non-limiting example, interaction with SIRPα. The antibodies are considered to completely modulate, block, inhibit, reduce, antagonize, neutralize or otherwise interfere with the CD47-SIRPα interaction when the level of CD47-SIRPα interaction in the presence of the antibody is decreased by at least 95%, e.g., by 96%, 97%, 98%, 99% or 100% as compared to the level of CD47-SIRPα interaction in the absence of binding with an antibody described herein. The antibodies are considered to partially modulate, block, inhibit, reduce, antagonize, neutralize or otherwise interfere with the CD47-SIRPα interaction when the level of CD47-SIRPα interaction in the presence of the antibody is decreased by less than 95%, e.g., 10%, 20%, 25%, 30%, 40%, 50%, 60%, 75%, 80%, 85% or 90% as compared to the level of CD47-SIRPα interaction in the absence of binding with an antibody described herein.

In some embodiments, the bispecific antibody exhibits a “balanced” affinity for each of the two targets. In other embodiments, the bispecific antibody exhibits an “unbalanced” affinity for each of the two targets. For example, in an anti-CD47/MSLN bispecific antibody, the affinity of the anti-MSLN arm is increased. For example, in an anti-CD47/MSLN bispecific antibody, the affinity of the anti-CD47 arm is decreased. For example, in an anti-CD47/MSLN bispecific antibody, the affinity of the anti-MSLN arm is increased and the affinity of the anti-CD47 arm is decreased. These unbalanced affinity bispecific antibodies are useful, for example, to improve selectivity for a target cell or group of target cells.

In some embodiments, the affinity of the anti-MSLN arm is increased by at least 100-fold following affinity maturation. In some embodiments, the affinity of the anti-CD47 arm is decreased by at least 2-fold following affinity dematuration. For example, in some embodiments, the anti-CD47 arm exhibits an affinity for CD47 that is between about 2-fold and 100-fold lower following affinity dematuration.

In some embodiments, the first arm amino acid sequence includes a variable heavy chain complementarity determining region 1 (CDRH1) comprising the amino acid sequence of SEQ ID NO: 225, a variable heavy chain complementarity determining region 2 (CDRH2) comprising the amino acid sequence of SEQ ID NO: 226, a variable heavy chain complementarity determining region 3 (CDRH3) comprising the amino acid sequence of SEQ ID NO: 227, a variable light chain complementarity determining region 1 (CDRL1) comprising the amino acid sequence of SEQ ID NO: 240, a variable light chain complementarity determining region 2 (CDRL2) comprising the amino acid sequence of SEQ ID NO: 242, and a variable light chain complementarity determining region 3 (CDRL3) comprising the amino acid sequence of SEQ ID NO: 254.

In some embodiments, the first arm amino acid sequence includes a variable heavy chain amino comprising the acid sequence of SEQ ID NO: 114 and a variable light chain comprising the amino acid sequence of SEQ ID NO: 168.

In some embodiments, the first arm amino acid sequence includes a heavy chain amino comprising the acid sequence of SEQ ID NO: 2 and a variable light chain comprising the amino acid sequence of SEQ ID NO: 56.

In some embodiments, the second arm amino acid sequence includes a variable heavy chain amino acid sequence of SEQ ID NO: 114 and a variable light chain amino acid sequence of SEQ ID NO: 222.

In some embodiments, the second arm amino acid sequence includes a heavy chain amino comprising the acid sequence of SEQ ID NO: 2 and a light chain comprising the amino acid sequence of SEQ ID NO: 108.

In some embodiments, the bispecific antibody includes two copies of a single heavy chain polypeptide and a first light chain and a second light chain, wherein the first and second light chains are different.

In some embodiments, at least a portion of the first light chain is of the Kappa type and at least a portion of the second light chain is of the Lambda type. In some embodiments, the first light chain includes at least a Kappa constant region. In some embodiments, the first light chain further includes a Kappa variable region. In some embodiments, the first light chain further includes a Lambda variable region. In some embodiments, the second light chain includes at least a Lambda constant region. In some embodiments, the second light chain further includes a Lambda variable region. In some embodiments, the second light chain further includes a Kappa variable region. In some embodiments, the first light chain includes a Kappa constant region and a Kappa variable region, and wherein the second light chain includes a Lambda constant region and a Lambda variable region.

In some embodiments, the constant and variable framework region sequences are human.

The disclosure also relates to the method of treating or preventing a disease by the administration of a bispecific antibody in combination with a second antibody. In some embodiments, the second antibody administered in combination with the bispecific antibody is a anti-PD-1 antibody. In some embodiments, the anti-PD-1 antibody is pembrolizumab.

The bispecific antibodies of the invention are generated using any methods known in the art such as, by way of non-limiting example, the use of cross-linked fragments, quadromas, and/or any of a variety of recombinant formats such as, by way of non-limiting examples, linked antibody fragments, forced heterodimers, and or recombinant formats based on single domains. Examples of Bispecific formats include but are not limited to bispecific IgG based on Fab arm exchange (Gramer et al., 2013 MAbs. 5(6)); the CrossMab format (Klein C et al., 2012 MAbs 4(6)); multiple formats based on forced heterodimerization approaches such as SEED technology (Davis J H et al., 2010 Protein Eng Des Sel. 23(4):195-202), electrostatic steering (Gunasekaran K et al., J Biol Chem. 2010 285(25):19637-46.) or knob-into-hole (Ridgway J B et al., Protein Eng. 1996 9(7):617-21.) or other sets of mutations preventing homodimer formation (Von Kreudenstein T S et al., 2013 MAbs. 5(5):646-54.); fragment based bispecific formats such as tandem scFv (such asBiTEs) (Wolf E et al., 2005 Drug Discov. Today 10(18):1237-44.); bispecific tetravalent antibodies (Pörtner L M et al., 2012 Cancer Immunol Immunother. 61(10):1869-75.); dual affinity retargeting molecules (Moore P A et al., 2011 Blood. 117(17):4542-51), diabodies (Kontermann R E et al., Nat Biotechnol. 1997 15(7):629-31).

In some embodiments, the bispecific antibodies carry a different specificity in each combining site and including two copies of a single heavy chain polypeptide and a first light chain and a second light chain, wherein the first and second light chains are different. In some embodiments, at least a first portion of the first light chain is of the Kappa type and at least a portion of the second light chain is of the Lambda type. In some embodiments, the first light chain includes at least a Kappa constant region. In some embodiments, the first light chain further includes a Kappa variable region. In some embodiments, the first light chain further includes a Lambda variable region. In some embodiments, the second light chain includes at least a Lambda constant region. In some embodiments, the second light chain further includes a Lambda variable region. In some embodiments, the second light chain further includes a Kappa variable region. In some embodiments, the first light chain includes a Kappa constant region and a Kappa variable region, and the second light chain includes a Lambda constant region and a Lambda variable region. In some embodiments, the constant and variable framework region sequences are human.

The bispecific antibodies of the invention that recognize MSLN and CD47 are generated using any methods known in the art such as, by way of non-limiting example, the use of cross-linked fragments, quadromas, and/or any of a variety of recombinant formats such as, by way of non-limiting examples, linked antibody fragments, forced heterodimers, and or recombinant formats based on single domains. The invention allows for the identification, production and purification of bispecific antibodies that are undistinguishable in sequence from standard antibodies and where one of the binding sites is specific for MSLN and the second binding site is specific for another target, for example a tumor-associated antigen (TAA). The unmodified nature of the antibodies of the invention provides them with favorable manufacturing and biochemical characteristics similar to standard monoclonal antibodies.

In some embodiments, the bispecific antibodies carry a different specificity in each combining site and including two copies of a single heavy chain polypeptide and a first light chain and a second light chain, wherein the first and second light chains are different.

In some embodiments, at least a first portion of the first light chain is of the Kappa type and at least a portion of the second light chain is of the Lambda type. In some embodiments, the first light chain includes at least a Kappa constant region. In some embodiments, the first light chain further includes a Kappa variable region. In some embodiments, the first light chain further includes a Lambda variable region. In some embodiments, the second light chain includes at least a Lambda constant region. In some embodiments, the second light chain further includes a Lambda variable region. In some embodiments, the second light chain further includes a Kappa variable region. In some embodiments, the first light chain includes a Kappa constant region and a Kappa variable region, and the second light chain includes a Lambda constant region and a Lambda variable region. In some embodiments, the constant and variable framework region sequences are human.

The monoclonal, monovalent and/or bispecific antibodies of the invention can be used for therapeutic intervention or as a research or diagnostic reagent. For example, the monoclonal, monovalent and/or bispecific antibodies of the invention are useful in methods of treating, preventing and/or delaying the progression of pathologies associated with aberrant CD47 and/or aberrant CD47-SIRPα expression and/or activity or alleviating a symptom associated with such pathologies, by administering an antibody of the invention to a subject in which such treatment or prevention is desired. The subject to be treated is, e.g., human. The monoclonal, monovalent and/or bispecific antibody is administered in an amount sufficient to treat, prevent, delay the progression or alleviate a symptom associated with the pathology.

In some embodiments, the monoclonal, monovalent and/or bispecific antibodies of the disclosure are useful in methods of treating, preventing and/or delaying the progression of, or alleviating a symptom of cancer or other neoplastic condition by administering an antibody of the invention to a subject in which such treatment or prevention is desired. For example, the monoclonal, monovalent and/or bispecific antibodies described herein are useful in treating hematological malignancies and/or solid tumors. For example, the monoclonal, monovalent and/or bispecific antibodies described herein are useful in treating CD47⁺ tumors, mesothelin⁺ tumors, and combinations thereof. By way of non-limiting example, the monoclonal, monovalent and/or bispecific antibodies described herein are useful in treating non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer, ovarian cancer, head and neck cancer, bladder cancer, melanoma, mesothelioma, colorectal cancer, cholangiocarcinoma, pancreatic cancer, including pancreatic adenocarcinoma, lung cancer, including lung adenocarcinoma, leiomyoma, leiomyosarcoma, kidney cancer, glioma, glioblastoma, endometrial cancer, esophageal cancer, biliary gastric cancer, and prostate cancer. Solid tumors include, e.g., breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

In certain embodiments, the monoclonal, monovalent, and/or bispecific antibodies of the disclosure are useful in methods of treating, preventing, and/or delaying the progression of, or alleviating a symptom of epithelial ovarian cancer, endometroid ovarian cancer, triple negative breast cancer, non-squamous non-small cell lung cancer, and/or pancreatic adenocarcinoma.

In some embodiments, the monoclonal, monovalent and/or bispecific antibodies described herein are used in conjunction with one or more additional agents or a combination of additional agents. Suitable additional agents include current pharmaceutical and/or surgical therapies for an intended application, such as, for example, cancer, inflammation and/or autoimmune diseases. In some embodiments, the monoclonal, monovalent and/or bispecific antibodies can be used in conjunction with rituximab.

In some embodiments, the monoclonal, monovalent, and/or bispecific antibodies described herein are used in conjunction with one or more additional agents or combination of agents. In certain embodiments, the agent is an anti-PD1 antibody. In certain embodiments, the agent is pembrolizumab.

In certain embodiments, the monoclonal, monovalent, and/or bispecific antibodies of the disclosure and combination pembrolizumab are useful in methods of treating, preventing, and/or delaying the progression of, or alleviating a symptom of epithelial ovarian cancer, endometroid ovarian cancer, triple negative breast cancer, non-squamous non-small cell lung cancer, and/or pancreatic adenocarcinoma. In certain embodiments, the patient has epithelial ovarian cancer, endometroid ovarian cancer, triple negative breast cancer, non-squamous non-small cell lung cancer, and/or pancreatic adenocarcinoma that is resistant to the standard of care therapy alone.

In some embodiments, the monoclonal, monovalent and/or bispecific antibodies and the additional agent are formulated into a single therapeutic composition, and the monoclonal, monovalent and/or bispecific antibody and additional agent are administered simultaneously. Alternatively, the monoclonal, monovalent and/or bispecific antibodies and additional agent are separate from each other, e.g., each is formulated into a separate therapeutic composition, and the monoclonal, monovalent and/or bispecific antibody and the additional agent are administered simultaneously, or the monoclonal, monovalent and/or bispecific antibodies and the additional agent are administered at different times during a treatment regimen. For example, the monoclonal, monovalent and/or bispecific antibody is administered prior to the administration of the additional agent, the monoclonal, monovalent and/or bispecific antibody is administered subsequent to the administration of the additional agent, or the monoclonal, monovalent and/or bispecific antibody and the additional agent are administered in an alternating fashion. As described herein, the monoclonal, monovalent and/or bispecific antibody and additional agent are administered in single doses or in multiple doses.

Pathologies treated and/or prevented using the antibodies of the invention include, for example, cancer or any other disease or disorder associated with aberrant CD47 expression and/or activity.

Pharmaceutical compositions according to the invention can include an antibody of the invention and a carrier. These pharmaceutical compositions can be included in kits, such as, for example, diagnostic kits.

### BRIEF DESCRIPTION OF THE DRAWINGS

FIG. 1 illustrates LCB-1801-001 study design. The trial is conducted in two parts (Parts A and B), each consisting of three phases.

FIG. 2 illustrates the LCB-1801-001 flow chart. The dose escalation phase begins (dose level 1) with a starting dose of 15 mg and ends (dose level 7) with a dose of 2000 mg. The dose expansion phase consists of expansion at the phase II recommended dose of 900 mg every two weeks. Indications include MSLN+ tumors such as platinum resistant ovarian cancer patients with at least 4 previous chemotherapy lines of treatment, triple negative breast cancer patients that have exhausted all approved standard of care treatments, and non-small cell lung cancer patients that have exhausted all approved standard of care treatments.

FIG. 3 illustrates NI-1801 in combination with anti-PD-1 antibody (i.e., pembrolizumab) flow chart. Dose limiting toxicities were cleared for dose levels 1-3 (beginning with a 300 mg at dose level 1 and ending with a 900 mg (300 mg loading dose) at dose level 3 in combination with the approved dose of pembrolizumab) and the dose expansion phase continues at dose level 3 with ten ovarian cancer, pancreatic cancer, and non-small cell lung cancer patients.

FIGS. 4A-4B illustrate the schedule of events for Part A and Part B. FIG. 4A shows the schedule of events for study entry, study drug administration, and safety assessment. FIG. 4B shows the schedule of events for response assessment and central PK and biomarker assessments. ADA, anti-drug antibodies; AE, adverse event; C, cycle; CT, computed tomography; D, day; ECG, electrocardiogram; ECHO, echocardiography; ECOG, Eastern Cooperative Oncology Group; EOT, end of treatment; LVEF, left ventricular ejection fraction; IV, intravenous; MUGA, multi-gated acquisition; PD, pharmacodynamic; PK, pharmacokinetic; PS, performance status. Note: All cycles are 28 days in duration, there are no rest periods between cycles. *For patients in the dose escalation phase (Part A) only. ^{#}If not done within the last 30 days as part of a treatment period, tumor assessment should also be performed at the EOT visit. ^{a}The informed consent consists of three parts. ^{b}Only patients enrolled in the sub-study will be treated with NI-1801 in combination with pembrolizumab. Pembrolizumab will be administered at the dosage of 400 mg every 6 weeks, in 4 cycles. Pembrolizumab will be administered as first drug; later, NI-1801 will be infused after 30 minutes. ^{c}Must include pulse and systolic/diastolic blood pressure as well as body temperature. Blood pressure has to be measured after resting for 5 minutes in a sitting position. ^{d}Serum β-hCG (for all female of childbearing potential [FCBP]) is performed at screening; remaining pregnancy tests may be serum or urine at the Investigator's discretion. Pregnancy testing (for all FCBP) must be done within 72 hours prior to the first administration of NI-1801 and prior to dosing on Day 1 of every cycle, and at the EOT visit. If the serum screening pregnancy test is performed >72 hours before first dose, a serum or urine pregnancy test should be performed (Investigator's discretion). The subject may not receive NI-1801 until the Investigator has verified that the result of the pregnancy test is negative. ^{h}Triplicate ECGs (i.e., 3 recordings within 2 [±1] minute intervals) will be performed at Screening and during NI-1801 treatment. ⁱFor patients with relevant tumor types, tumor marker (CA-125 or CYFRA 21-1 as relevant) will be checked during screening and thereafter as indicated. ^{j}CT scans or MRI to include chest, abdomen, and pelvis. Measurements using revised RECIST 1.1. At screening and thereafter until intolerable toxicity or progressive disease as indicated. ^{k}Testing for MSLN expression necessary for study entrance can be performed using archival tumor tissue and is foreseen to be performed at the institution's pathology. Mandatory slice for centralized assessment for validation analysis.

### DETAILED DESCRIPTION

CD47 or Integrin-Associated-Protein (IAP) is a ubiquitous 50 kDa transmembrane glycoprotein with multiple functions in cell-cell communication. It interacts with multiple ligands, such as, for example, integrins, and/or SIRPα. In the context of the innate immune system, CD47 functions as a marker of self, transmitting an inhibitory “don't kill me” signal through binding to SIRPα expressed by myeloid cells, such as macrophages, neutrophils, and dendritic cells. The role of widespread expression of CD47 in the physiological situation is therefore to protect healthy cells against the elimination by the innate immune system (Oldenborg P A, et al., CD47-Signal Regulatory Protein α (Sirpα) Regulates Fcγ and Complement Receptor-Mediated Phagocytosis, J Exp Med. 2001 Apr. 2; 193(7):855-62; Mattias Olsson, Role of the CD47/SIRPα-interaction in regulation of macrophage phagocytosis, Department of Integrative Medical Biology, Section for Histology and Cell Biology, Umea University, Umeå, Sweden, Thesis; Oldenborg P A., Role of CD47 in erythroid cells and in autoimmunity, Leuk Lymphoma. 2004 July; 45(7):1319-27; Oldenborg P A, et al., Role of CD47 as a Marker of Self on Red Blood Cells., Science. 2000 Jun. 16; 288(5473):2051-4; Brown E J, Frazier W A., integrin-associated protein (CD47) and its ligands., Trends Cell Biol. 2001 March; 11(3):130-5).

Tumor cells hijack this immunosuppressive mechanism by overexpressing CD47, which efficiently helps them to escape immune surveillance and killing by innate immune cells. (Majeti R, Chet al., CD47 is an adverse prognostic factor and therapeutic antibody target on human acute myeloid leukemia stem cells, Cell. 2009 Jul. 23; 138(2):286-99; S. Jaiswal et al., CD47 is upregulated on circulating hematopoietic stem cells and leukemia cells to avoid phagocytosis., Cell. 2009 Jul. 23; 138(2):271-85). CD47 expression is upregulated in most human cancers (e.g., NHL, AML, breast, colon, glioblastoma, glioma, ovarian, bladder and prostate cancers) and increased levels of CD47 expression clearly correlate with aggressive disease and poor survival. (Majeti R, et al., Cell. 2009 Jul. 23; 138(2):286-99; S. Jaiswal et al., Cell. 2009 Jul. 23; 138(2):271-85; Willingham S B, et al., The CD47-signal regulatory protein alpha (SIRPα) interaction is a therapeutic target for human solid tumors, Proc Natl Acad Sci USA. 2012 Apr. 24; 109(17):6662-7; Chao M P, et al., Therapeutic antibody targeting of CD47 eliminates human acute lymphoblastic leukemia, Cancer Res. 2011 Feb. 15; 71(4):1374-84).

The widespread expression of CD47 in healthy tissues brings the question of treatment safety and efficacy: First, targeting CD47 with a neutralizing monoclonal antibody (Mab) could affect healthy cells, resulting in severe toxicities as shown in preclinical studies with mice and cynomolgus monkeys (Willingham S B, et al., Proc Natl Acad Sci USA. 2012 Apr. 24; 109(17):6662-7; Weiskopf K, et al., Engineered SIRPα Variants as Immunotherapeutic Adjuvants to Anticancer Antibodies, Science. 2013 Jul. 5; 341(6141):88-91). Second, even if severe toxicities could be avoided or mitigated by using alternative formats (Weiskopf K, et al., Science. 2013 Jul. 5; 341(6141):88-91), broad expression of CD47 could still cause a rapid elimination of CD47-binding molecules through target-mediated drug disposition resulting in poor pharmacokinetics and decreased efficacy.

The bispecific antibody compositions were described in U.S. Pat. No. 11,260,117 (the contents of which are incorporated by reference in their entireties).

CD47 (Cluster of Differentiation 47) functions as a “don't eat me” signal for phagocytic cells and is known to be over-expressed by many tumors (immune escape). CD47 interacts with SIRPα, which is expressed on phagocytic cells. CD47 down-regulates phagocytic activity. CD47 inhibits dendritic cell (DC) maturation and activation. CD47 has also been implicated in processes such as, for example, apoptosis, survival, proliferation, adhesion, migration, and regulation of angiogenesis, blood pressure, tissue perfusion, and/or platelet homeostasis.

CD47 has also been implicated in cancer. For example, CD47 is overexpressed in various hematological and solid malignancies. CD47 is a documented cancer stem cell/tumor initiating cell marker. It is thought that CD47 overexpression may help tumor cells to escape immune surveillance and killing by innate immune cells. High levels of CD47 are also associated with poor clinical outcome in cancers such as, for example, leukemias, lymphomas, breast cancer, colon cancer, ovarian cancer, bladder cancer, prostate cancer, and/or glioma. Thus, targeting CD47 would be useful in treating, delaying the progression of, or otherwise ameliorating a symptom of cancer.

Mesothelin (MSLN) is expressed in normal tissues at relatively low levels. In contrast to normal tissues, mesothelin is highly expressed in several types of solid tumors such as malignant mesothelioma, ovarian cancer, pancreatic adenocarcinoma, lung adenocarcinoma, as well as endometrial, biliary gastric and prostate cancers. Tumor mesothelin expression has often been correlated with increased tumor aggressiveness and poor clinical outcome. Thus, targeting mesothelin would be useful in treating, delaying the progression of, or otherwise ameliorating a symptom of cancer.

Mesothelin (MSLN) is a 40 kDa glycosylphosphatidylinositol (GPI)-linked cell surface glycoprotein that is generated proteolytically from a 71 kDa precursor. In normal tissues, mesothelin is expressed—at relatively low levels—only in mesothelial cells lining serosal membranes such as the pleura, peritoneum, and pericardium. The normal physiologic function of mesothelin remains unclear but it seems dispensable, since mesothelin deficient mice grow and reproduce normally and display no obvious abnormalities.

In contrast to normal tissues, mesothelin is highly expressed in several types of solid tumors such as malignant mesothelioma, ovarian cancer, pancreatic adenocarcinoma, lung adenocarcinoma, as well as endometrial, biliary gastric and prostate cancers. Tumor mesothelin expression has often been correlated with increased tumor aggressiveness and poor clinical outcome. Mesothelin binding to ovarian cancer antigen MUC16 (CA-125) has been shown to mediate cell-to-cell adhesion, possibly contributing metastatic dissemination. In addition, mesothelin-mediated intracellular signaling was reported to promote tumor cell proliferation, as well as resistance to chemotherapy and to anoikis (programmed cell death resulting from loss of normal cell-matrix interactions).

Similar to most other GPI-anchored proteins, mesothelin is shed from the membrane, and soluble mesothelin has been reported in sera of tumor patients. Soluble mesothelin is therefore a useful biomarker, for diagnosis of mesothelin-positive tumors, but also for monitoring disease progression and response to treatment. Soluble mesothelin is also considered as a negative prognostic biomarker for patients with ovarian cancer, lung or pancreatic adenocarcinoma, and triple-negative breast cancer. Last but not least, serum mesothelin levels are a predictive biomarker in mesothelioma, as they have been found to positively correlate with therapeutic responses to mesothelin-targeting therapies.

Most tumor-associated antigens used to therapeutically target solid tumors are also expressed in essential normal tissues. In contrast, expression of mesothelin is generally low-level and limited to mesothelial cells (which seem dispensable). On the other hand, cell-surface expression of mesothelin is high in many solid tumors, which makes mesothelin a particularly attractive target of therapeutic intervention. Accordingly, numerous mesothelin-directed therapies, using monoclonal antibodies, recombinant immunotoxins, antibody-drug conjugates, cancer vaccines, and chimeric antigen receptor T cells, are currently under development, including clinical evaluation at late-stage trials for MPM and pancreatic adenocarcinoma.

The invention also provides bispecific antibodies that recognize CD47 and mesothelin.

The bispecific antibodies of the invention allow for simultaneous binding of the two antibody arms to two antigens on the surface of the cell (termed co-engagement), which results in additive or synergistic increase of affinity due to avidity mechanism. Consequently, co-engagement confers high selectivity towards cells expressing both antigens as compared to cells that express just one single antigen. In addition, the affinities of the two arms of a bispecific antibody to their respective targets can be set up in a way that binding to target cells is principally driven by one of the antibody arms. In some embodiments, the bispecific antibody includes a first arm that binds CD47 and a second arm that binds mesothelin, where the second arm binds to mesothelin with high affinity, and the first arm binds to CD47 with low affinity, i.e., an affinity that is sufficient to inhibit CD47/SIRPα upon mesothelin co-engagement. This design allows the bispecific antibodies of the invention to preferentially inhibit CD47 in cancer versus normal cells. In the examples provided herein, a bispecific antibody with a first arm that binds CD47 with low affinity and a second arm that binds MSLN with high affinity (termed a CD47×MSLN bispecific) allow preferential inhibition of CD47 in cancer versus normal cells. Besides the two antigen-binding arms, the CD47×MSLN bispecific antibody requires a functional Fc portion to recruit macrophages and/or other immune effector cells. A fully human bispecific IgG format (such as the κλ-body format described herein) is well suited for the generation of dual targeting CD47×MSLN bispecific antibodies. The ability of dual targeting bispecific antibodies to co-engage CD47 and MSLN results in efficient and selective cancer cell killing mediated by the CD47×MSLN bispecific antibody, as demonstrated in the ADCC and ADCP experiments provided herein.

In some embodiments, exemplary bispecific antibodies of the invention that include at least a first arm that binds CD47 include a combination of heavy chain and light chain complementarity determining regions (CDRs) selected from the CDR sequences shown in Tables 1, 2 and 3, where the CDRs shown in Tables 1, 2 and 3 are defined according to the IMGT nomenclature.

In some embodiments, exemplary bispecific antibodies of the invention that include at least a first arm that binds CD47 include the combination of heavy chain CDR sequences from Table 1 and two sets of light chain CDRs selected from the CDRL1, CDRL2 and CDRL3 sequences shown in Tables 2 and 3.

In some embodiments, exemplary bispecific antibodies of the invention that include at least a first arm that binds CD47 include the combination of heavy chain CDR sequences from Table 1 and a first set of light chain CDRs selected from the CDRL1, CDRL2 and CDRL3 sequences shown in Table 2 and a second set of light chain CDRs selected from the CDRL1, CDRL2 and CDRL3 sequences shown in Table 3.

In some embodiments, exemplary bispecific antibodies of the invention that include a first arm that binds CD47 and a second arm that binds MSLN, wherein the first arm includes the combination of heavy chain complementarity determining regions (CDRs) shown in Table 1 and a combination of the light chain CDRs selected from the CDR sequences shown in Table 2, and wherein the second arm includes the combination of heavy chain complementarity determining regions (CDRs) shown in Table 1 and a combination of the light chain CDRs selected from the CDR sequences shown in Table 3.

TABLE 1
Common Heavy Chain CDRs

| CDRH1 | CDRH2 | CDRH3 |
|---|---|---|
| GFTFSSYA | ISGSGGST | AKSYGAFDY |
| (SEQ ID NO: 225) | (SEQ ID NO: 226) | (SEQ ID NO: 227) |

TABLE 2
Anti-CD47 Kappa Light Chain CDRs

| CDRL1 | CDRL2 | CDRL3 |
|---|---|---|
| QSISSY | AAS | QQMHPRAPKT |
| (SEQ ID NO: 240) | (SEQ ID NO: 242) | (SEQ ID NO: 254) |

TABLE 3
Anti-MSLN Lambda Light Chain CDRs

| CDRL1 | CDRL2 | CDRL3 |
|---|---|---|
| SGINVRDYR | YKSASDK | MIWHHDSEGHAFV |
| (SEQ ID NO: 286) | (SEQ ID NO: 292) | (SEQ ID NO: 298) |

Each of the exemplary anti-CD47, anti-MSLN, monovalent and bispecific antibodies described herein include a common heavy chain (HC), one kappa chain or one lambda chain for anti-CD47 and anti-MSLN antibodies, one kappa and one lambda light chains (LC) for monovalent and bispecific antibodies, as shown in the amino acid and corresponding nucleic acid sequences listed below. Each of the exemplary anti-CD47, anti-MSLN, monovalent and bispecific antibodies described below includes a common variable heavy domain (VH), one kappa variable light domain or one lambda variable light domain for anti-CD47 and anti-MSLN antibodies, one kappa and one lambda variable light domains (VL) for monovalent and bispecific antibodies, as shown in the amino acid and corresponding nucleic acid sequences listed below.

While antibody sequences below are provided herein as examples, it is to be understood that these sequences can be used to generate bispecific antibodies using any of a variety of art-recognized techniques. Examples of bispecific formats include but are not limited to bispecific IgG based on Fab arm exchange (Gramer et al., 2013 MAbs. 5(6)); the CrossMab format (Klein C et al., 2012 MAbs 4(6)); multiple formats based on forced heterodimerization approaches such as SEED technology (Davis J H et al., 2010 Protein Eng Des Sel. 23(4):195-202), electrostatic steering (Gunasekaran K et al., J Biol Chem. 2010 285(25):19637-46.) or knob-into-hole (Ridgway J B et al., Protein Eng. 1996 9(7):617-21.) or other sets of mutations preventing homodimer formation (Von Kreudenstein T S et al., 2013 MAbs. 5(5):646-54.); fragment based bispecific formats such as tandem scFv (such asBiTEs) (Wolf E et al., 2005 Drug Discov. Today 10(18):1237-44.); bispecific tetravalent antibodies (Portner L M et al., 2012 Cancer Immunol Immunother. 61(10):1869-75.); dual affinity retargeting molecules (Moore P A et al., 2011 Blood. 117(17):4542-51), diabodies (Kontermann R E et al., Nat Biotechnol. 1997 15(7):629-31).

The exemplary anti-CD47, anti-MSLN, monovalent and bispecific antibodies include a common heavy chain (SEQ ID NO: 2) encoded by the nucleic acid sequence shown in SEQ ID NO: 1.

>COMMON-HC-NT
(SEQ ID NO: 1)
GAGGTGCAGCTGTTGGAGTCTGGGGGAGGCTTGGTACAGCCTGGGGGGTCCCTGAGACTCTCCTGTG
CAGCCTCTGGATTCACCTTTAGCAGCTATGCCATGAGCTGGGTCCGCCAGGCTCCAGGGAAGGGGCT
GGAGTGGGTCTCAGCTATTAGTGGTAGTGGTGGTAGCACATACTACGCAGACTCCGTGAAGGGCCGG
TTCACCATCTCCAGAGACAATTCCAAGAACACGCTGTATCTGCAAATGAACAGCCTGAGAGCCGAGG
ACACGGCCGTATATTACTGTGCGAAAAGTTATGGTGCTTTTGACTACTGGGGCCAGGGAACCCTGGT
CACAGTCTCGAGCGCCTCCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAACCGGTGACAGTCTCGT
GGAACTCAGGAGCCCTGACCAGCGGCGTGCACACCTTCCCGGCTGTCCTACAGTCCTCAGGACTCTA
CTCCCTCAGCAGCGTGGTGACTGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACA
CATGCCCACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAAGCCGC
GGGAGGAGCAGTACAACAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTATACCCTGCCCCCATCTCGGGAGGAGATGA
CCAAGAACCAGGTCAGCCTGACTTGCCTGGTCAAAGGCTTCTATCCCAGCGACATCGCCGTGGAGTG
GGAGAGCAACGGGCAGCCGGAGAACAACTACAAGACCACGCCTCCCGTGCTGGACTCCGACGGCTCC
TTCTTCCTCTATAGCAAGCTCACCGTGGACAAGTCCAGGTGGCAGCAGGGGAACGTCTTCTCATGCT
CCGTGATGCATGAGGCTCTGCACAACCACTACACGCAGAAGAGCCTCTCCCTGTCTCCGGGTTAA
>COMMON-HC-AA
(SEQ ID NO: 2)
EVQLLESGGGLVQPGGSLRLSCAASGFTFSSYAMSWVRQAPGKGLEWVSAISGSGGSTYYADSVKGR
FTISRDNSKNTLYLQMNSLRAEDTAVYYCAKSYGAFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPG

The anti-CD47, anti-MSLN, monovalent and bispecific antibodies include a common variable heavy domain (SEQ ID NO: 114) encoded by the nucleic acid sequence shown in SEQ ID NO: 113.

>COMMON-VH-NT
(SEQ ID NO: 113)
GAGGTGCAGCTGTTGGAGTCTGGGGGAGGCTTGGTACAGCCTGGGGGGTCCCTGAGACTCTCCTGTG
CAGCCTCTGGATTCACCTTTAGCAGCTATGCCATGAGCTGGGTCCGCCAGGCTCCAGGGAAGGGGCT
GGAGTGGGTCTCAGCTATTAGTGGTAGTGGTGGTAGCACATACTACGCAGACTCCGTGAAGGGCCGG
TTCACCATCTCCAGAGACAATTCCAAGAACACGCTGTATCTGCAAATGAACAGCCTGAGAGCCGAGG
ACACGGCCGTATATTACTGTGCGAAAAGTTATGGTGCTTTTGACTACTGGGGCCAGGGAACCCTGGT
CACAGTCTCGAGC
>COMMON-VH-AA
(SEQ ID NO: 114)
EVQLLESGGGLVQPGGSLRLSCAASGFTFSSYAMSWVRQAPGKGLEWVSAISGSGGSTYYADSVKGR
FTISRDNSKNTLYLQMNSLRAEDTAVYYCAKSYGAFDYWGQGTLVTVSS

### Anti-CD47 Antibodies

The Ka3 antibody includes a common heavy chain (SEQ ID NO: 2) encoded by the nucleic acid sequence shown in SEQ ID NO: 1 and includes a kappa light chain (SEQ ID NO: 56) encoded by the nucleic acid sequence shown in SEQ ID NO: 55.

>KA3-LC-NT
(SEQ ID NO: 55)
GACATCCAGATGACCCAGTCTCCATCCTCCCTGTCTGCATCTGTAGGAGACAGAGTCACCATCACTT
GCCGGGCAAGTCAGAGCATTAGCAGCTATTTAAATTGGTATCAGCAGAAACCAGGGAAAGCCCCTAA
GCTCCTGATCTATGCTGCATCCAGTTTGCAAAGTGGGGTCCCATCAAGGTTCAGTGGCAGTGGATCT
GGGACAGATTTCACTCTCACCATCAGCAGTCTGCAACCTGAAGATTTTGCAACTTACTACTGTCAGC
AGATGCACCCGCGCGCCCCGAAGACCTTCGGCCAAGGGACCAAGGTGGAAATCAAACGTACGGTGGC
TGCACCATCTGTCTTCATCTTCCCGCCATCTGATGAGCAGTTGAAATCTGGAACTGCCTCTGTTGTG
TGCCTGCTGAATAACTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTACAGCCTCAGCAGCAC
CCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAA
>KA3-LC-AA
(SEQ ID NO: 56)
DIQMTQSPSSLSASVGDRVTITCRASQSISSYLNWYQQKPGKAPKLLIYAASSLQSGVPSRFSGSGS
GTDFTLTISSLQPEDFATYYCQQMHPRAPKTFGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQG
LSSPVTKSFNRGEC

The Ka3 antibody includes a common variable heavy domain (SEQ ID NO: 114) encoded by the nucleic acid sequence shown in SEQ ID NO: 113 and includes a kappa variable light domain (SEQ ID NO: 168) encoded by the nucleic acid sequence shown in SEQ ID NO: 167.

>KA3-VL-NT
(SEQ ID NO: 167)
GACATCCAGATGACCCAGTCTCCATCCTCCCTGTCTGCATCTGTAGGAGACAGAGTCACCATCACTT
GCCGGGCAAGTCAGAGCATTAGCAGCTATTTAAATTGGTATCAGCAGAAACCAGGGAAAGCCCCTAA
GCTCCTGATCTATGCTGCATCCAGTTTGCAAAGTGGGGTCCCATCAAGGTTCAGTGGCAGTGGATCT
GGGACAGATTTCACTCTCACCATCAGCAGTCTGCAACCTGAAGATTTTGCAACTTACTACTGTCAGC
AGATGCACCCGCGCGCCCCGAAGACCTTCGGCCAAGGGACCAAGGTGGAAATCAAA
>KA3-VL-AA
(SEQ ID NO: 168)
DIQMTQSPSSLSASVGDRVTITCRASQSISSYLNWYQQKPGKAPKLLIYAASSLQSGVPSRFSGSGS
GTDFTLTISSLQPEDFATYYCQQMHPRAPKTFGQGTKVEIK

### Anti-Mesothelin (Anti-MSLN) Antibodies

The 038 antibody includes a common heavy chain (SEQ ID NO: 2) encoded by the nucleic acid sequence shown in SEQ ID NO: 1 and includes a lambda light chain (SEQ ID NO: 108) encoded by the nucleic acid sequence shown in SEQ ID NO: 107. The variable region of the lambda light chain is bolded in the amino acid sequence below.

>038-LC-NT
(SEQ ID NO: 107)
CAGCCTGTGCTGACTCAGCCGGCTTCCCTCTCTGCATCTCCTGGGGCATCAGCCAGTCTCACCTGCA
CCTTGCGCAGTGGCATCAACGTTAGAGATTACAGGATATTCTGGTACCAGCAGAAGCCAGGGAGTCC
TCCCCAGTATCTCCTGAGGTACAAAAGCGCATCAGATAAGCAGCAGGGCTCTGGAGTCCCCAGCCGC
TTCTCTGGGTCCAAAGATGCTTCGGCCAATGCAGGGATTTTACTCATCTCTGGGCTCCAGTCTGAGG
ATGAGGCTGACTATTACTGTATGATTTGGCACCACGATTCGGAGGGGCATGCTTTTGTGTTCGGCGG
AGGGACCAAGCTGACCGTCCTAGGTCAGCCCAAGGCTGCCCCCTCGGTCACTCTGTTCCCGCCCTCC
TCTGAGGAGCTTCAAGCCAACAAGGCCACACTGGTGTGTCTCATAAGTGACTTCTACCCGGGAGCCG
TGACAGTGGCTTGGAAAGCAGATAGCAGCCCCGTCAAGGCGGGAGTGGAGACCACCACACCCTCCAA
ACAAAGCAACAACAAGTACGCGGCCAGCAGCTATCTGAGCCTGACGCCTGAGCAGTGGAAGTCCCAC
AGAAGCTACAGCTGCCAGGTCACGCATGAAGGGAGCACCGTGGAGAAGACAGTGGCCCCTACAGAAT
GTTCATAA
>038-LC-AA
(SEQ ID NO: 108)
QPVLTQPASLSASPGASASLTCTLRSGINVRDYRIFWYQQKPGSPPQYLLRYKSASDKQQGSGVPSR
FSGSKDASANAGILLISGLQSEDEADYYCMIWHHDSEGHAFVFGGGTKLTVLGQPKAAPSVTLFPPS
SEELQANKATLVCLISDFYPGAVTVAWKADSSPVKAGVETTTPSKQSNNKYAASSYLSLTPEQWKSH
RSYSCQVTHEGSTVEKTVAPTECS

The 038 antibody includes a common variable heavy domain (SEQ ID NO: 114) encoded by the nucleic acid sequence shown in SEQ ID NO: 113 and includes a lambda variable light domain (SEQ ID NO: 222) encoded by the nucleic acid sequence shown in SEQ ID NO: 221.

>038-VL-NT
(SEQ ID NO: 221)
CAGCCTGTGCTGACTCAGCCGGCTTCCCTCTCTGCATCTCCTGGGGCATCAGCCAGTCTCACCTGCA
CCTTGCGCAGTGGCATCAACGTTAGAGATTACAGGATATTCTGGTACCAGCAGAAGCCAGGGAGTCC
TCCCCAGTATCTCCTGAGGTACAAAAGCGCATCAGATAAGCAGCAGGGCTCTGGAGTCCCCAGCCGC
TTCTCTGGGTCCAAAGATGCTTCGGCCAATGCAGGGATTTTACTCATCTCTGGGCTCCAGTCTGAGG
ATGAGGCTGACTATTACTGTATGATTTGGCACCACGATTCGGAGGGGCATGCTTTTGTGTTCGGCGG
AGGGACCAAGCTGACCGTCCTA
>038-VL-AA
(SEQ ID NO: 222)
QPVLTQPASLSASPGASASLTCTLRSGINVRDYRIFWYQQKPGSPPQYLLRYKSASDKQQGSGVPSR
FSGSKDASANAGILLISGLQSEDEADYYCMIWHHDSEGHAFVFGGGTKLTVL

### Dummy Light Chains

The Dummy light chain 1 (SEQ ID NO: 112) is encoded by the nucleic acid sequence shown in SEQ ID NO: 111.

>DUMMY-LC1-NT
(SEQ ID NO: 111)
CAGTCTGTGTTGACGCAGCCGCCCTCAGTGTCTGCGGCCCCAGGACAGAAGGTCACCATCTCCTGCT
CTGGAAGCAGCTCCAATATTGAGACTGGTTCTGTATCCTGGTACCAGCAGCTCCCAGGAACAGCCCC
CAAACTCCTCATTTATGACAATAATAAGCGACCCTCAGGGATTCCTGACCGATTCTCTGGCTCCAAG
TCTGGCACGTCAGCCACCCTGGGCATCACCGGACTCCAGACTGGGGACGAGGCCGATTATTACTGCG
GAACATGGGATGACAGCCTGCCTGGATGGGTGTTCGGCGGAGGGACCAAGCTGACCGTCCTAGGTCA
GCCCAAGGCTGCCCCCTCGGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCTTGGAAAGCAGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCATAA
>DUMMY-LC1-AA
(SEQ ID NO: 112)
QSVLTQPPSVSAAPGQKVTISCSGSSSNIETGSVSWYQQLPGTAPKLLIYDNNKRPSGIPDRFSGSK
SGTSATLGITGLQTGDEADYYCGTWDDSLPGWVFGGGTKLTVLGQPKAAPSVTLFPPSSEELQANKA
TLVCLISDFYPGAVTVAWKADSSPVKAGVETTTPSKQSNNKYAASSYLSLTPEQWKSHRSYSCQVTH
EGSTVEKTVAPTECS

The Dummy variable light domain 1 (SEQ ID NO: 206) is encoded by the nucleic acid sequence shown in SEQ ID NO: 205.

>DUMMY-VL1-NT
(SEQ ID NO: 205)
CAGTCTGTGTTGACGCAGCCGCCCTCAGTGTCTGCGGCCCCAGGACAGAAGGTCACCATCTCCTGCT
CTGGAAGCAGCTCCAATATTGAGACTGGTTCTGTATCCTGGTACCAGCAGCTCCCAGGAACAGCCCC
CAAACTCCTCATTTATGACAATAATAAGCGACCCTCAGGGATTCCTGACCGATTCTCTGGCTCCAAG
TCTGGCACGTCAGCCACCCTGGGCATCACCGGACTCCAGACTGGGGACGAGGCCGATTATTACTGCG
GAACATGGGATGACAGCCTGCCTGGATGGGTGTTCGGCGGAGGGACCAAGCTGACCGTCCTA
>DUMMY-VL1-AA
(SEQ ID NO: 206)
QSVLTQPPSVSAAPGQKVTISCSGSSSNIETGSVSWYQQLPGTAPKLLIYDNNKRPSGIPDRFSGSK
SGTSATLGITGLQTGDEADYYCGTWDDSLPGWVFGGGTKLTVL

The Dummy light chain 2 (SEQ ID NO: 208) is encoded by the nucleic acid sequence shown in SEQ ID NO: 207.

>DUMMY-LC2-NT
(SEQ ID NO: 207)
GAAATAGTGATGACGCAGTCTCCAGCCACCCTGTCTGTGTCTCCAGGGGAAAGAGCCACCCTCTCCT
GCAGGGCCAGTCAGACGGTTAAGAATAATTTAGCCTGGTACCAGCAGAAACCTGGCCAGGCTCCCAG
GCTCCTCATCTATGGTGCATCCACCAGGGCCACTGGTATCCCAGCCAGGTTCAGTGGCAGTGGGTCT
GGGACAGAGTTCACTCTCACCATCAGCAGCCTGCAGTCTGAAGATTTTGCAGTTTATTACTGTCAGC
AGTATAACAACTGGTTGCCCATCAACCCCTATACCTTCGGCCAAGGGACCAAGGTGGAAATCAAACG
TACGGTGGCTGCACCATCTGTCTTCATCTTCCCGCCATCTGATGAGCAGTTGAAATCTGGAACTGCC
TCTGTTGTGTGCCTGCTGAATAACTTCTATCCCAGAGAGGCCAAAGTACAGTGGAAGGTGGATAACG
CCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTAA
>DUMMY-LC2-AA
(SEQ ID NO: 208)
EIVMTQSPATLSVSPGERATLSCRASQTVKNNLAWYQQKPGQAPRLLIYGASTRATGIPARFSGSGS
GTEFTLTISSLQSEDFAVYYCQQYNNWLPINPYTFGQGTKVEIKRTVAAPSVFIFPPSDEQLKSGTA
SVVCLLNNFYPREAKVQWKVDNALQSGNSQESVTEQDSKDSTYSLSSTLTLSKADYEKHKVYACEVT
HQGLSSPVTKSFNRGEC

The Dummy variable light domain 2 (SEQ ID NO: 210) is encoded by the nucleic acid sequence shown in SEQ ID NO: 209.

>DUMMY-VL2-NT
(SEQ ID NO: 209)
GAAATAGTGATGACGCAGTCTCCAGCCACCCTGTCTGTGTCTCCAGGGGAAAGAGCCACCCTCTCCT
GCAGGGCCAGTCAGACGGTTAAGAATAATTTAGCCTGGTACCAGCAGAAACCTGGCCAGGCTCCCAG
GCTCCTCATCTATGGTGCATCCACCAGGGCCACTGGTATCCCAGCCAGGTTCAGTGGCAGTGGGTCT
GGGACAGAGTTCACTCTCACCATCAGCAGCCTGCAGTCTGAAGATTTTGCAGTTTATTACTGTCAGC
AGTATAACAACTGGTTGCCCATCAACCCCTATACCTTCGGCCAAGGGACCAAGGTGGAAATCAAA
>DUMMY-VL2-AA
(SEQ ID NO: 210)
EIVMTQSPATLSVSPGERATLSCRASQTVKNNLAWYQQKPGQAPRLLIYGASTRATGIPARFSGSGS
GTEFTLTISSLQSEDFAVYYCQQYNNWLPINPYTFGQGTKVEIK

### Bispecific Antibody

In some embodiments, the bispecific antibody Ka3×O38 includes a common heavy chain comprising a CDRH1 comprising the amino acid sequence of SEQ ID NO: 225, a CDRH2 comprising the amino acid sequence of SEQ ID NO: 226, a CDRH3 comprising the amino acid sequence of SEQ ID NO: 227, a kappa light chain comprising a CDRL1 comprising the amino acid sequence of SEQ ID NO: 240, a CDRL2 comprising the amino acid sequence of SEQ ID NO: 242, and a CDRL3 comprising the amino acid sequence of SEQ ID NO: 254, and a lambda light chain comprising a CDRL1 comprising the amino acid sequence of SEQ ID NO: 286, a CDRL2 comprising the amino acid sequence of SEQ ID NO: 292, and a CDRL3 comprising the amino acid sequence of SEQ ID NO: 298.

In some embodiments, the bispecific antibody Ka3×O38 includes a common heavy chain variable region (SEQ ID NO: 114) encoded by the nucleic acid sequence shown in SEQ ID NO: 113, a kappa light chain variable region (SEQ ID NO: 168) encoded by the nucleic acid sequence shown in SEQ ID NO: 167, and a lambda light chain variable region (SEQ ID NO: 222) encoded by the nucleic acid sequence shown in SEQ ID NO: 221.

In some embodiments, the bispecific antibody Ka3×O38 includes a common heavy chain (SEQ ID NO: 2) encoded by the nucleic acid sequence shown in SEQ ID NO: 1, a kappa light chain (SEQ ID NO: 56) encoded by the nucleic acid sequence shown in SEQ ID NO: 55, and a lambda light chain (SEQ ID NO: 108) encoded by the nucleic acid sequence shown in SEQ ID NO: 109.

### Methods of Treatment Using Ka3×O38 (NI-1801)

The disclosure herein provides for a method of method of treating or preventing cancer in a patient in need thereof, comprising administering to the patient a therapeutically effective amount of a bispecific antibody comprising a first arm that comprises a first amino acid sequence that binds CD47 and a second arm that comprises a second amino acid that binds mesothelin (MSLN).

In some embodiments, the bispecific antibody is administered as a single agent, that is, without an additional combination therapy. In some embodiments, the bispecific antibody is administered in combination with another therapeutic agent, such as a second antibody. In some embodiments, the bispecific antibody and the second antibody are administered at different therapeutically effective doses and at different schedules, respectively.

In some embodiments, the patient is treated with a therapeutically effective amount of an anti-CD47×anti-MSLN bispecific antibody and a therapeutically effective amount of an anti-PD-1 antibody (i.e., a combination therapy).

In certain embodiments, the bispecific antibody is Ka3×O38 or a fragment thereof. Ka3×O38 refers to “NI-1801”.

In typical embodiments, the bispecific antibody comprises a heavy chain comprising a CDRH1 comprising the amino acid sequence of SEQ ID NO: 225, a CDRH2 comprising the amino acid sequence of SEQ ID NO: 226, a CDRH3 comprising the amino acid sequence of SEQ ID NO: 227, a kappa light chain comprising a CDRL1 comprising the amino acid sequence of SEQ ID NO: 240, a CDRL2 comprising the amino acid sequence of SEQ ID NO: 242, and a CDRL3 comprising the amino acid sequence of SEQ ID NO: 254, and a lambda light chain comprising a CDRL1 comprising the amino acid sequence of SEQ ID NO: 286, a CDRL2 comprising the amino acid sequence of SEQ ID NO: 292, and a CDRL3 comprising the amino acid sequence of SEQ ID NO: 298, wherein the therapeutically effective amount of the bispecific antibody is about 15 mg to about 1200 mg.

In some embodiments, the bispecific antibody comprises a variable heavy chain comprising the amino acid of SEQ ID NO: 114 and a variable kappa light chain comprising the amino acid sequence of SEQ ID NO: 168 and a variable lambda light chain comprising the amino acid sequence of SEQ ID NO: 222.

In some embodiments, the bispecific antibody comprises a heavy chain comprising the amino acid sequence of SEQ ID NO: 2, a kappa light chain comprising the amino acid sequence of SEQ ID NO: 56, and a lambda light chain comprising the amino acid sequence of SEQ ID NO: 108.

In some embodiments, the patient is further administered a therapeutically effective amount of a monoclonal antibody or fragment thereof that binds PD-1. In typical embodiments, the monoclonal antibody that binds PD-1 is pembrolizumab.

In typical embodiments, pembrolizumab is administered at an FDA approved dose. In some embodiments, the therapeutically effective amount of the monoclonal antibody is about 400 mg.

In some embodiments, the bispecific antibody is administered by intravenous injection. In some embodiments, pembrolizumab is administered by intravenous injection. In some embodiments, the bispecific antibody and pembrolizumab are administered by intravenous injection.

In some embodiments, the any one of the bispecific antibody or pembrolizumab are administered to the patient over the course of a 1-hour or 2-hour infusion.

In some embodiments, the bispecific antibody is administered as a pharmaceutical composition comprising at least one pharmaceutically acceptable carrier. In some embodiments, the monoclonal antibody is administered as a pharmaceutical composition comprising at least one pharmaceutically acceptable carrier.

In certain embodiments, the bispecific antibody is administered once weekly.

In certain embodiments, the bispecific antibody is administered once weekly for at least one cycle, wherein a cycle is 28 days. In some embodiments, the bispecific antibody is administered once weekly for at least six cycles.

In some embodiments, the bispecific antibody is administered once weekly for at least one cycle, wherein a cycle is 28 days and the monoclonal antibody pembrolizumab is administered every six weeks for at least 4 cycles, wherein a cycle is 28 days.

In certain embodiments, the bispecific antibody is administered once every 2 weeks.

In certain embodiments, the bispecific antibody is administered once every 2 weeks for at least one cycle, wherein a cycle is 28 days. In some embodiments, the bispecific antibody is administered once every 2 weeks for at least six cycles.

In some embodiments, the bispecific antibody is administered once every 2 weeks for at least one cycle, wherein a cycle is 28 days and the monoclonal antibody pembrolizumab is administered once every six weeks for at least 4 cycles, wherein a cycle is 28 days.

In some embodiments, the therapeutically effective amount of the bispecific antibody is about 15 mg to about 50 mg. In some embodiments, the therapeutically effective amount of the bispecific antibody is about 50 mg to about 150 mg. In some embodiments, the therapeutically effective amount of the bispecific antibody is about 150 mg to about 300 mg. In some embodiments, the therapeutically effective amount of the bispecific antibody is about 300 mg to about 450 mg. In some embodiments, the therapeutically effective amount of the bispecific antibody is about 450 mg to about 600 mg. In some embodiments, the therapeutically effective amount of the bispecific antibody is about 600 mg to about 750 mg. In some embodiments, the therapeutically effective amount of the bispecific antibody is about 750 mg to about 900 mg. In some embodiments, the therapeutically effective amount of the bispecific antibody is about 900 mg to about 1200 mg. In some embodiments, the therapeutically effective amount of the bispecific antibody is about 1200 mg to about 2000 mg. In some embodiments, the therapeutically effective amount of the bispecific antibody is about 300 mg. In some embodiments, the therapeutically effective amount of the bispecific antibody is about 600 mg. In some embodiments, the therapeutically effective amount of the bispecific antibody is about 900 mg. In some embodiments, the therapeutically effective amount of the bispecific antibody is about 2000 mg.

In typical embodiments, the patient in need of a therapeutically effective amount of the bispecific antibody as a single agent has cancer. In certain embodiments, the patient in need of a therapeutically effective amount of a combination of the bispecific antibody and a therapeutically effective amount of the anti-PD-1 antibody has cancer.

In some embodiments, the cancer is a solid tumor.

In some embodiments, the cancer expresses mesothelin (MSLN). In some embodiments, the solid tumor is or is derived from ovarian cancer, triple-negative breast cancer (TNBC), non-squamous non-small cell lung cancer (NCSLC), head and neck cancer, bladder cancer, melanoma, mesothelioma, colorectal cancer, cholangiocarcinoma, pancreatic cancer, leiomyoma, leiomyosarcoma, kidney cancer, glioma, glioblastoma, endometrial cancer, esophageal cancer, biliary gastric cancer, prostate cancer, or combinations thereof.

In some embodiments, patients that have triple-negative breast cancer are administered the bispecific antibody as a sole agent without any combination therapy of anti-PD-1 antibody.

In certain embodiments, the solid tumor is or is derived from epithelial ovarian cancer, endometroid ovarian cancer, triple negative breast cancer, non-squamous non-small cell lung cancer, and/or pancreatic adenocarcinoma. In certain embodiments, the solid tumor is resistant to the standard of care therapy alone.

In certain embodiments, the subject is an adult greater than 18 years of age.

In some embodiments, before administration of any one of the antibodies disclosed herein to a patient, said patient has a histologically or cytologically confirmed diagnosis of any one of epithelial ovarian cancer, triple negative breast cancer, or non-squamous non-small cell lung cancer. In certain embodiments, the patient has a MSLN positive tumor, wherein MSLN expression is confirmed with a staining intensity of >2+ as per immunohistochemistry (IHC) in >40% of tumor cells.

In some embodiments, prior to administration of any one of the antibodies disclosed herein to a patient, said patient has a confirmed diagnosis of advanced, metastatic, or recurrent cancer.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, F_{ab}, F_{ab′} and F_{(ab′)2} fragments, scFvs, and an F_{ab} expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is the to specifically bind an antigen when the dissociation constant is ≤1 μM; e.g., ≤100 nM, preferably ≤10 nM and more preferably ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is the to specifically bind to its target, when the equilibrium binding constant (K_{d}) is ≤1 μM, e.g., ≤100 nM, preferably ≤10 nM, and more preferably ≤1 nM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules, and nucleic acid molecules encoding the light chain immunoglobulin molecules described herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules, and the light chain immunoglobulin molecules described herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

As used herein, the twenty conventional amino acids and their abbreviations follow conventional usage. See Immunology—A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, σ-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

### Antibodies

Various procedures known within the art may be used for the production of polyclonal or monoclonal antibodies directed against a given target, such as, for example, CD47, a tumor associated antigen or other target, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, NY, incorporated herein by reference).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

In some embodiments, the antibodies of the invention are monoclonal antibodies. Monoclonal antibodies are generated, for example, by using the procedures set forth in the Examples provided herein. Antibodies are also generated, e.g., by immunizing BALB/c mice with combinations of cell transfectants expressing high levels of a given target on their surface. Hybridomas resulting from myeloma/B cell fusions are then screened for reactivity to the selected target.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Monoclonal antibodies of the invention include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies are antibody molecules in which the entire sequence of both the light chain and the heavy chain, including the CDRs, arise from human genes. Such antibodies are termed “human antibodies”, or “fully human antibodies” herein. Monoclonal antibodies can be prepared by using trioma technique; the human B-cell hybridoma technique (see Kozbor, et al., 1983 Immunol Today 4: 72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds specifically to the relevant epitope with high affinity are disclosed in PCT publication WO 99/53049.

The antibody can be expressed by a vector containing a DNA segment encoding the single chain antibody described above.

These can include vectors, liposomes, naked DNA, adjuvant-assisted DNA. gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

Bispecific antibodies are antibodies that have binding specificities for at least two different antigens. In the present case, one of the binding specificities is for a target such as CD47 or any fragment thereof. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Bispecific and/or monovalent antibodies of the invention can be made using any of a variety of art-recognized techniques, including those disclosed in co-pending application WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This r-k-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof. (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in co-pending application WO 2012/023053 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in co-pending applications WO 2010/135558 and WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. The bispecific antibodies of the invention can be of different Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies of the invention. (see for example Strohl, W R Curr Opin Biotechnol 2009 (6):685-91; U.S. Pat. No. 6,528,624; PCT/US2009/0191199 filed Jan. 9, 2009). The methods of the invention can also be used to generate bispecific antibodies and antibody mixtures in a F(ab′)2 format that lacks the Fc portion.

The common heavy chain and two different light chains are co-expressed into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ubc (Human ubiquitin C promoter); SV40 (Simian virus 40 promoter). Different IRES have also been described from mammalian and viral origin. (See e.g., Hellen C U and Samow P. Genes Dev 2001 15: 1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97: 1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in co-pending application PCT/IB2012/003028, filed on Oct. 19, 2012, published as WO2013/088259, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in treating cancer and/or other diseases and disorders associated with aberrant CD47 expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

### Use of Anti-CD47×Anti-MSLN Antibodies

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include an antibody of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as CD47, mesothelin, or a combination thereof (or a fragment thereof), may be used in methods known within the art relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the invention can be used to isolate a particular target using standard techniques, such as immunoaffinity, chromatography or immunoprecipitation. Antibodies of the invention (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds. For example, the antibody binds to the target and neutralizes or otherwise inhibits the interaction between CD47 and SIRPα.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., F_{ab}, scFv, or F_{(ab)2}) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### EXEMPLARY EMBODIMENTS

Exemplary Embodiment No. 1. A method of treating or preventing cancer in a patient in need thereof, comprising administering to the patient a therapeutically effective amount of a bispecific antibody comprising a first arm that comprises a first amino acid sequence that binds CD47 and a second arm that comprises a second amino acid that binds mesothelin (MSLN);
wherein the bispecific antibody comprises a heavy chain comprising a CDRH1 comprising the amino acid sequence of SEQ ID NO: 225, a CDRH2 comprising the amino acid sequence of SEQ ID NO: 226, a CDRH3 comprising the amino acid sequence of SEQ ID NO: 227,
a kappa light chain comprising a CDRL1 comprising the amino acid sequence of SEQ ID NO: 240, a CDRL2 comprising the amino acid sequence of SEQ ID NO: 242, and a CDRL3 comprising the amino acid sequence of SEQ ID NO: 254, and
a lambda light chain comprising a CDRL1 comprising the amino acid sequence of SEQ ID NO: 286, a CDRL2 comprising the amino acid sequence of SEQ ID NO: 292, and a CDRL3 comprising the amino acid sequence of SEQ ID NO: 298; and
wherein the therapeutically effective amount of the bispecific antibody is about 15 mg to about 1200 mg.

Exemplary Embodiment No. 2. The method of the previous Exemplary Embodiments, wherein the bispecific antibody comprises a variable heavy chain comprising the amino acid of SEQ ID NO: 114 and a variable kappa light chain comprising the amino acid sequence of SEQ ID NO: 168 and a variable lambda light chain comprising the amino acid sequence of SEQ ID NO: 222.

Exemplary Embodiment No. 3. The method of claim 1 or 2, wherein the bispecific antibody comprises a heavy chain comprising the amino acid sequence of SEQ ID NO: 2, a kappa light chain comprising the amino acid sequence of SEQ ID NO: 56, and a lambda light chain comprising the amino acid sequence of SEQ ID NO: 108.

Exemplary Embodiment No. 4. The method of any one of claims 1-3, further administering to the patient a therapeutically effective amount of a monoclonal antibody that binds PD-1.

Exemplary Embodiment No. 5. The method of claim 4, wherein the monoclonal antibody that binds PD-1 is pembrolizumab.

Exemplary Embodiment No. 6. The method of any one of claims 4-5, wherein the therapeutically effective amount of the monoclonal antibody is about 400 mg.

Exemplary Embodiment No. 7. The method of any one of claims 1-3, wherein the bispecific antibody is administered by intravenous injection.

Exemplary Embodiment No. 8. The method of any one of claims 4-6, wherein the monoclonal antibody is administered by intravenous injection.

Exemplary Embodiment No. 9. The method of claim 7, wherein the bispecific antibody is administered as a pharmaceutical composition comprising at least one pharmaceutically acceptable carrier.

Exemplary Embodiment No. 10. The method of claim 8, wherein the monoclonal antibody is administered as a pharmaceutical composition comprising at least one pharmaceutically acceptable carrier.

Exemplary Embodiment No. 11. The method of claim 9, wherein the administration is once weekly.

Exemplary Embodiment No. 12. The method of claim 11, wherein the bispecific antibody is administered once weekly for at least one cycle, wherein a cycle is 28 days.

Exemplary Embodiment No. 13. The method of claim 12, wherein the bispecific is administered once weekly for at least six cycles.

Exemplary Embodiment No. 14. The method of claim 10, further comprising administering the monoclonal antibody every six weeks for at least 4 cycles, wherein a cycle is 28 days.

Exemplary Embodiment No. 15. The method of any one of claims 1-14, wherein the therapeutically effective amount of the bispecific antibody is 15 mg to 50 mg.

Exemplary Embodiment No. 16. The method of any one of claims 1-15, wherein the therapeutically effective amount of the bispecific antibody is 50 mg to 150 mg.

Exemplary Embodiment No. 17. The method of any one of claims 1-15, wherein the therapeutically effective amount of the bispecific antibody is 150 mg to 300 mg.

Exemplary Embodiment No. 18. The method of any one of claims 1-15, wherein the therapeutically effective amount of the bispecific antibody is 300 mg to 450 mg.

Exemplary Embodiment No. 19. The method of any one of claims 1-15, wherein the therapeutically effective amount of the bispecific antibody is 450 mg to 600 mg.

Exemplary Embodiment No. 20. The method of any one of claims 1-15, wherein the therapeutically effective amount of the bispecific antibody is 600 mg to 750 mg.

Exemplary Embodiment No. 21. The method of any one of claims 1-15, wherein the therapeutically effective amount of the bispecific antibody is 750 mg to 900 mg.

Exemplary Embodiment No. 22. The method of any one of claims 1-15, wherein the therapeutically effective amount of the bispecific antibody is 900 mg to 1200 mg.

Exemplary Embodiment No. 23. The method of any one of claims 1-22, wherein the cancer is a solid tumor.

Exemplary Embodiment No. 24. The method of any one of claims 1-23, wherein the cancer expresses MSLN.

Exemplary Embodiment No. 25. The method of any one of claims 1-24, wherein the solid tumor is or is derived from ovarian cancer, triple-negative breast cancer (TNBC), non-squamous non-small cell lung cancer (NCSLC), head and neck cancer, bladder cancer, melanoma, mesothelioma, colorectal cancer, cholangiocarcinoma, pancreatic cancer, leiomyoma, leiomyosarcoma, kidney cancer, glioma, glioblastoma, endometrial cancer, esophageal cancer, biliary gastric cancer, prostate cancer, or combinations thereof.

### EXAMPLES

### Example 1: Nonclinical Pharmacokinetics and Drug Metabolism

The nonclinical pharmacokinetics (PK) and drug metabolism of NI-1801 (i.e, Ka3×O38) was determined. The PK of NI-1801 was investigated in both anon-cross-reactive species, mouse (C57BL/6J), and in a cross-reactive species, monkey (cynomolgus). While NI-1801 can bind both human and cynomolgus monkey CD47 and MSLN with similar affinities, no cross-reactivity was reported in mice.

The PK profile of NI-1801 was evaluated in C57BL/6J mice following a single IV injection of 5 mg/kg. As a comparator, a separate group of mice was administered a single IV injection of 5 mg/kg of a control human IgG1 bsAb, which has been shown to have a liner pharmacokinetic profile in the same non-cross-reactive species.

The terminal half-life (t_{1/2}) of NI-1801 was 329 hours, i.e., 13.7 days. Previous reports have shown that IgGs bind to the neonatal Fc receptor (FcRn) which reduces their catabolism by receptor-mediated endocytosis (pinocytosis) leading to a long half-life in vivo. Moreover, human IgG1 has been described to bind mouse FcRn with superior affinity than mouse IgG, thus explaining the relative long elimination half-life of human IgG1 in this species.

In summary, NI-1801 bsAb has a PK profile like that of human IgG1 bsAb control and exhibited a biphasic and linear elimination profile. No unexpected off-target binding was identified and the ability to bind mouse FcRn was equivalent to the control human IgG1 bsAb.

### Single and Repeat-Dose Pharmacokinetics

The PK profile of NI-1801 was investigated in male and female cynomolgus monkeys. NI-1801 was administered as a single-dose to one male and one female per group at doses of 0.5 or 10 mg/kg and as repeat doses (on Days 1, 8, 15, and 22) to one male and two females at 10 mg/kg. NI-1801 was administered via IV bolus at a dose of 1 mL/kg.

Following single dose administration of NI-1801, T_{1/2} of NI-1801 was estimated in females to be approx. 140 hours (5.8 days) at 0.5 mg/kg and 99 hours (4.1 days) at 10 mg/kg, respectively. For male animals (both dose-levels), due to the limited timepoints in the terminal phase, T_{1/2} could not be considered as appropriately estimated and reliable.

Differences between male and female monkeys were mainly seen in the terminal phase, and to a lesser extent in the distribution phase. Results showed comparable AUC_{0-168 h} and a lower AUC₀₋ₗₐₛₜ in males. After repeated dosing at 10 mg/kg, a slight to moderate decrease in AUC was observed in week 4 in comparison to week 1 for the three animals. This possible decrease in exposure after repeated dosing may be due to anti-CD47/MSLN antibodies (ADA).

### Toxicokinetics Dose Range Finding Study

A dose range finding (DRF) study was performed in cynomolgus monkeys to evaluate the toxicity and toxicokinetic profile of NI-1801. NI-1801 was administered bi-weekly by IV bolus over four weeks (i.e., seven administrations in total) to two male and two female monkeys at doses of 15 or 45 mg/kg, respectively.

NI-1801 exposure in treated animals was comparable in male and female cynomolgus monkeys. Systemic exposure to NI-1801 increased in a dose-proportional manner in the range of administered doses following Day 1 administration.

NI-1801 was quantifiable in serum samples on Day 1 from 0.25 hours to last timepoint (96 hours) prior to the subsequent dosing at both doses of 15 and 45 mg/kg. On Day 1, concentration profiles were similar between animals for the same dose and between sexes.

During week 4, the overall toxicokinetic profile was consistent between animals, with one exception, that is a female from the 15 mg/kg treatment group which had notably lower exposure (mainly from 4-8 hours post-dosing onwards) compared to the three remaining animals from the same group.

Following administration of NI-1801 at 15 mg/kg, the mean exposure values (males/females) for Cₘₐₓ and AUC_{0-168 h} were 481/490 μg/mL and 24′571/24′213 hours*μg/mL respectively, while at 45 mg/kg NI-1801, the mean exposure values (males/females) for Cₘₐₓ and AUC_{0-168 h} were 1′757/1′743 μg/mL and 169′725/144′727 hours*μg/mL, respectively, at the end of the treatment period.

4-Week Repeat-Dose Toxicokinetics from GLP Toxicity Study

The toxicokinetic profile of NI-1801 was investigated during the 4-week GLP toxicity study conducted in cynomolgus monkeys. During this study, NI-1801 was administered bi-weekly as a slow IV bolus, to three animals/sex/group (at 15 mg/kg dose) or five animals/sex/group (at 45 mg/kg dose). Animals received a total of seven injections over four weeks (i.e., Day 1, 5, 9, 13, 17, 21, and 25).

On Day 1, NI-1801 was quantifiable over the sampling interval (from 15 min to 96 hours post-dose) and at all doses. At 15 and 45 mg/kg/administration, the profiles showed a constant decline of the serum concentrations of NI-1801.

Following repeated administrations (Day 25), a significant decrease in exposure was observed after the 7^{th} and last dose in two females and two males (15 mg/kg/administration) which were also reported to be ADA positive. The high dose group animals (45 mg/kg/administration) showed quantifiable serum concentrations of NI-1801 until 96 hours post-dose after the last dose on Day 25. Serum concentrations in the 45 mg/kg/administration recovery animals remained quantifiable for up to 25 days (i.e., 4 weeks of recovery period or Study Day 50) after the end of treatment; except for one female where the last quantifiable concentration was detected on Day 36. This female was tested ADA positive on Day 50. Overall, no gender differences were seen between the male and females on the parameters measured. The AUC increased in a dose proportional manner. For ADA negative animals, NI-1801 was quantifiable up to 96 hours post-dose on both Day 1 and Day 25, with a slight increase in systemic exposure observed by Day 25, with accumulation ratios ranging from 1.40 to 3.13 for both males and females.

### Comparative Systemic Exposure Margins

Two population PK models were developed using data originating from studies in cynomolgus monkeys: One after the first injection to avoid the impact of ADAs on NI-1801 PK and a second one also including data after the last administration to describe NI-1801 PK in the presence of ADAs. The first model was used to simulate NI-1801 PK profiles in humans using allometric scaling.

The systemic exposure margins (or margins of safety) have been calculated using the exposures (Cₘₐₓ and AUC) in the 4-week repeat-dose toxicity studies in cynomolgus monkeys compared to predicted exposures in humans. Due to different designs between preclinical and clinical studies, the following comparisons were made: For Cₘₐₓ, in monkeys the first observed NI-1801 concentration after the last injection was considered as Cₘₐₓ (t+0.25 h after bolus administration), while in humans, NI-1801 concentration at the end of the infusion was considered as Cₘₐₓ (t+1 h after the start of the last infusion). For AUC in humans, AUC_{0-168 h} was computed based on simulated NI-1801 clearance after the last injection, while in monkeys the predicted NI-1801 clearance after the first injection was used to derive AUC_{inf}. This approach was considered as the more conservative in terms of exposures, avoiding the implication of immunogenicity in cynomolgus, which significantly decrease NI-1801 exposure in some animals.

Based on this comparison. the Cₘₐₓ and AUC values in patients dosed weekly for 4 weeks with NI-1801 at e.g., 150 mg are predicted to be at least 32-fold and 10.2-fold below those measured in cynomolgus monkeys at the 45 mg/kg/administration no-observed-adverse-effect level (NOAEL) dose. While at 600 mg, for example, the predicted human Cₘₐₓ and AUC values in patients should remain at least 8- and 2.5-fold below those measured in monkeys. Please refer to the Investigator's Brochure for a tabulated summary of the exposure margins.

### Example 2: Nonclinical Toxicology

The following section describes the nonclinical toxicology of NI-1801. The cynomolgus monkey was identified as the relevant species to investigate in vivo the safety of NI-1801 and therefore two studies were conducted in this species.

### 4-Week DRF Study in Cynomolgus Monkeys

NI-1801 was administered twice weekly by IV (bolus) over four weeks (seven administrations in total) to two male and two female cynomolgus monkeys at doses of 15 or 45 mg/kg/administration. Animals were observed daily for clinical signs, local tolerance, body weight, food consumption. Blood samples were collected for toxicokinetics, hematology, coagulation, and blood chemistry. Organ weights, gross pathology and histopathology were investigated upon sacrifice of the animals.

Following twice weekly administration of NI-1801 for up to four weeks, there were no test item-related effects on clinical signs, body weight or food consumption.

Clinical pathology findings were limited to decreases in white blood cell, neutrophil and lymphocyte counts observed in high-dose (45 mg/kg) females and one high-dose male, observed from Day 15 and at the end of the treatment period (Day 28) for almost all animals. Decreased platelet count was also noted in all animals at 45 mg/kg/administration at Day 28. None of these changes was considered as adverse. No effects were observed on coagulation and blood chemistry parameters.

There were no organ weight differences or related macroscopic findings. NI-1801-related non-adverse microscopic findings were noted in the lymphoid organs (spleen, lymph nodes and GALT), and consisted of decreased lymphoid cellularity (in germinal centers mainly).

The no-observed-adverse-effect level (NOAEL) was therefore set at 45 mg/kg/administration. At this dose level, the mean exposure values (males/females), namely Cₘₐₓ and AUC_{0-168 h}, were 1′757/1′743 μg/mL and 169′725/144′727 h*μg/mL at the end of the treatment period.

### 4-Week GLP Toxicology Study in Cynomolgus Monkeys

A 4-week study GLP repeat-dose study was conducted in cynomolgus monkeys to investigate the toxicity of NI-1801 following IV administrations at 15 or 45 mg/kg for four weeks, with an assessment of the reversibility of potential findings after a 6-week recovery (treatment free) period.

In this study, NI-1801 or vehicle were administered twice weekly on study days 1, 5, 9, 13, 17, 21, and 25 (every 96 hours) to 26 monkeys of Mauritian origin by slow bolus IV injections. Three animals/sex/group were terminated after four weeks of treatment and two animals/sex in the control and high-dose (i.e., 45 mg/kg) groups were terminated at the end of the recovery period.

Assessment of toxicity was based on clinical observations, body weights, food consumption, examination of injection sites, clinical pathology (hematology, clinical chemistry, and coagulation), ophthalmoscopies, urinalysis, and anatomic pathology examinations.

Following twice weekly administrations of NI-1801 for up to four weeks, no unscheduled deaths occurred, and no NI-1801-treatment related clinical signs were observed. Body weight, body weight gains, and food intake remained unaffected. No NI-1801-treatment related abnormalities were reported at ophthalmic examinations, urinalysis, and blood biochemistry investigations. Overall, NI-1801 treatment was clinically well tolerated.

Hematological changes were noted in males at >15 mg/kg/administration and females at 45 mg/kg/administration. They appeared from Day 2 onward and were limited to a minimal decrease in white blood cell counts compared to pre-study values, mainly triggered by neutrophils and lymphocytes decreased counts. These changes were aligned to those noted by immunophenotyping, at 45 mg/kg/administration, which consisted of decreases in absolute and relative B- and NK-cell counts. Fibrinogen levels were minimally increased in several individuals at ≥15 mg/kg/administration at the end of the treatment period. All changes were reversible following the recovery period except for the NK-cells counts, which tended towards recovery, but remained below pre-dose levels and 1.5 to 1.8-fold lower than in control animal.

There were no NI-1801-related macroscopic or organ weight changes at any dose level and microscopic findings were non-adverse and limited to those observed at 45 mg/kg/administration. This included a non-adverse decreased lymphoid cellularity in the lymph nodes (partially to fully reversible), spleen, and gut associated lymphoid tissue (fully reversible) and a non-adverse multisystemic arterial inflammation/necrosis and/or perivascular inflammatory cell infiltrate (minimal or slight). The latter was observed in a single male at the end of the treatment period and in both recovery females at the end of the dose-free period in the 45 mg/kg dose group and considered to be likely secondary to immunogenicity of NI-1801.

Microscopic findings observed in the injection sites in NI-1801-treated animals were similar to those seen in control animals and/or were as expected after repeated IV injection. Similarly, reactions observed at the IV injection sites after dosing (i.e., swelling of the injected cephalic vein, abnormal color and scabs around the injected area) were present in all groups, including vehicle control group and were, therefore, attributed to the IV injection procedure rather than NI-1801 treatment.

There were no acute or chronic NI-1801 related effects on hemodynamics, ECG, and respiratory parameters and no abnormalities were reported at neurobehavioral examinations at any dose-level.

Based on the absence of adverse findings at any dose level, the NOAEL for this study was identified at 45 mg/kg/administration.

### Human Tissue Cross-Reactivity Study

A GLP-compliant tissue cross-reactivity study was conducted to investigate the potential off-target cross-reactivity of NI-1801.

A validated immunohistochemistry (IHC) method using biotin-conjugated NI-1801 was applied for testing of 42 frozen human tissues and blood smears (three donors per tissue). Sections of ovarian carcinoma (endometrioid adenocarcinoma) were used as positive controls.

In human tissues, biotinylated NI-1801 produced staining in cells whose morphology and distribution were generally consistent with mesothelial cells (duodenum, jejunum, ileum, cecum, lung, ovary, spleen), in epithelial cells of the oviduct and in a subset of squamous epithelial cells in the tonsil, described in the literature to express MSLN. Staining was also seen in some other epithelial cells (breast, parotid gland, prostate, urinary bladder), where expression of one or both targets is plausible based on the ubiquitous expression of CD47, in rare capillary blood vessel walls of the spleen and of thyroid, and in cytoplasmic granules consistent with lipofuscin (adrenal, thyroid).

CD47 is ubiquitously expressed, although levels are heterogenous on various tissues and cells according to the literature. As such, the staining pattern observed in this study is assumed to represent on-target binding on tissues expressing both mesothelin and CD47.

The staining in breast was not deemed relevant as it was mainly cytoplasmic and may be due to overexpression of MSLN secondary to breast cancer. The staining in urinary bladder, adrenal, and thyroid (follicular cells), mucous cells was also not relevant as it was exclusively cytoplasmic or represented artefacts. For the results observed in the remaining tissues (i.e., capillary vessels in thyroid and spleen, parotid gland), for which published data on MSLN expression is currently insufficient or limited to mRNA expression, staining could not be definitively attributed to NI-1801 binding or artifacts, and as such, non-specific binding could not be ruled out.

### Example 3: First-In-Human Clinical Study in Patients with MSLN-Expressing Ovarian, Pancreatic, Non-Small Cell Lung, and Triple Negative Breast Cancer

Given non-clinical data showing significant anti-tumor effects (Example 1 and 2 above) NI-1801 (i.e., Ka3×O38) as new investigational medicinal product (IMP) has a strong biological rationale for the treatment of subjects with advanced, metastatic, or recurrent solid malignancies expressing MSLN.

NI-1801 is evaluated in this first-in-human (FIH) clinical study in patients with MSLN-expressing advanced, metastatic, or recurrent ovarian cancer (OC), triple negative breast cancer (TNBC), or non-small cell lung cancer (NSCLC), respectively, who have failed treatment with, are intolerant to, or are not candidates for available therapies that are known to confer a clinical benefit to patients with these tumor entities. This patient population represents an area of very high unmet clinical need.

The present study is designed to establish the safety, tolerability, PK, pharmacodynamics (PD), and preliminary efficacy of NI-1801 in patients with advanced, metastatic, or recurrent solid MSLN expressing malignancies. The study will be conducted in two parts: dose escalation (Part A) and cohort expansion (Part B).

Pembrolizumab is used as cancer medicine used to treat NSCLC and other cancers, authorized in Europe in 2015. Pembrolizumab, is a monoclonal antibody, a protein that has been designed to recognize and block a receptor (‘target’) called PD-1. Some cancers can make a protein (PD-L1) that combines with PD-1 to switch off the activity of certain cells of the immune system (the body's natural defenses), preventing them from attacking the cancer. By blocking PD-1, pembrolizumab stops the cancer switching off these immune cells, thereby increasing the immune system's ability to kill the cancer cells.

### Study Design Rationale

In Part A and in the combination with pembrolizumab cohort, a modified accelerated titration design will be used to establish initial safety and tolerability and identify the recommended dose for cohort expansion in Part B. Additional safety and efficacy information will be obtained in Part B for a larger cohort with up to 10 patients in total.

### Starting Dose and Dosing Schedule Rationale

The starting dose for this trial is 15 mg NI-1801 administered as a 1-hour infusion. This dose is based on the NOAEL determined in a 4-week dose range finding (DRF) general toxicity study as well as a 4-week GLP toxicology study in cynomolgus monkeys as most appropriate species (Examples above). For the latter assessment, besides the general inter-species comparability, the cross-reactivity of NI-1801 with cynomolgus CD47 and MSLN was considered pivotal. No non-severe or severe effects on hematological parameters in cynomolgus such as RBCs and platelet counts up to 45 mg/kg/administration were observed. With a conversion factor of 3.1, this value corresponds to a human equivalent dose of 14.5 mg/kg. With a safety factor of 10, this relates to a dose of 1.45 mg/kg body weight. Related to the planned starting dose of 15 mg flat dose, this corresponds to an additional safety factor of 6.8 (for a 70 kg individual), or a total safety factor of 68. For higher body weights, the safety factor increases. Due to lack of cross-reactivity between human and murine CD47 as well as MSLN, mouse is not a relevant species for NOAEL-determination. In in vitro assays, there was no apparent platelet aggregation or hemagglutination event at concentrations ranging from 0.2 to 200 μg/mL and 0.1 to 200 μg/mL, respectively. There was likewise no cytokine release observed following single or repeated IV injection of NI-1801 in monkeys. The margins of safety have been calculated using the exposures (Cₘₐₓ and AUC) in the 4-week repeat-dose toxicity studies in cynomolgus monkeys compared to predicted exposures in humans. Based on this comparison, the Cₘₐₓ and AUC values in patients dosed weekly for four weeks with NI-1801 at 15 mg are predicted to be at least 318-fold and 102-fold below those measured in cynomolgus monkeys at the 45 mg/kg NOAEL dose.

The starting dose is a fixed dose of 15 mg per subject.

Furthermore, in order to safeguard patient safety, following Cohort 5, all patients receive in Cycle 1 (Day 1) a starting dose of NI 1801 of 300 mg, followed by a subsequent dose, as defined by the SRC in the dose escalation cohorts or the recommended dose in the expansion cohort. The above applies to the single agent and the combination with pembrolizumab cohorts.

The starting NI-1801 dose of 300 mg is considered as safe and tolerable as per SRC. Pembrolizumab will be administered at the dosage of 400 mg every 6 weeks, in 4 cycles. Pembrolizumab will be administered as first drug; later, NI-1801 will be infused after 30 minutes.

As NI-1801 is used in this trial for the first time in man, no prior toxicity data in humans are available. Expected toxicities can be related to those observed in a trial using a structurally comparable molecule targeting CD47 and CD19 on B-cell malignancies. Given the comparable mechanism of action, a similar toxicity profile is likely to be encountered. In this case, the dosing schedule for NI-1801 starts with four dose levels under the MTD observed at 500 mg, i.e., three cohorts with one individual each, and one dose level (i.e., 300 mg) with three to six individuals foreseen.

Subjects will be dosed once weekly. The projected T_{1/2} in humans is approx. 7 days, supporting a QW (or longer) dosing interval. The increased length in dosing interval is supported by the anticipation of a decrease in the number MSLN positive target cells over time with continual dosing.

### Benefit/Risk Assessment

The patient population eligible for this FIH clinical study (LCB-1801-001) is those subjects with advanced malignancies which are resistant, refractory, or intolerant to standard therapy that is known to confer a clinical benefit, or those without standard therapy and in whom, in the opinion of the Investigator, experimental therapy with NI-1801 is beneficial. The life expectancy in most of these subjects is expected to be at least 8 weeks, and NI-1801 dosing is for up to 6 months until confirmed disease progression, unacceptable toxicity, or subject/Investigator decision to withdraw. NI-1801 treatment can extend beyond 6 cycles for those patients who do not have disease progression.

Evidence on pembrolizumab shows that the drug is effective in delaying worsening of the disease and improving survival in patients with NSCLC that tested positive for the PD-L1 protein.

As NI-1801 is used in this trial for the first time in man, no prior toxicity data in humans are available. Based on the preclinical safety and toxicology profiles, NI-1801 is considered to have demonstrated an acceptable benefit-risk ratio.

Toxicities observed in a trial using a structurally comparable molecule targeting CD47 and CD19 on B-cell malignancies (TG-1801; NCT03804996, NCT04806035) referred the following: Here, 11 subjects with B-cell lymphomas were treated with TG-1801 with median follow-up of <6 months. Five dose levels were tested: 20 mg (1 subject), 60 mg (1 subject), 180 mg (1 subject), 360 mg (4 subjects), and 500 mg (4 subjects). Observed toxicities included thrombocytopenia (3 subjects), anemia, diarrhea, fatigue, neutropenia, and IRRs (1 subject each). Two subjects had a DLT in the 500 mg dose level cohort. One subject in this cohort had grade 4 thrombocytopenia and one had inability to resume TG-1801 dosing within 14 days of stopping drug due to IRRs. Consequently, the 500 mg dose level exceeded the MTD defined by the protocol. As per study design, the 360 mg dose level cohort is being expanded to a total of 6 evaluable subjects.

Pembrolizumab is authorized in Europe for treating various types of cancer and the most common side effects when used alone include feeling tired, pain, including pain in muscles, rash, diarrhea, fever, cough, decreased appetite, itching, shortness of breath, constipation, bones or joints and stomach-area (abdominal) pain, nausea, and low levels of thyroid hormone.

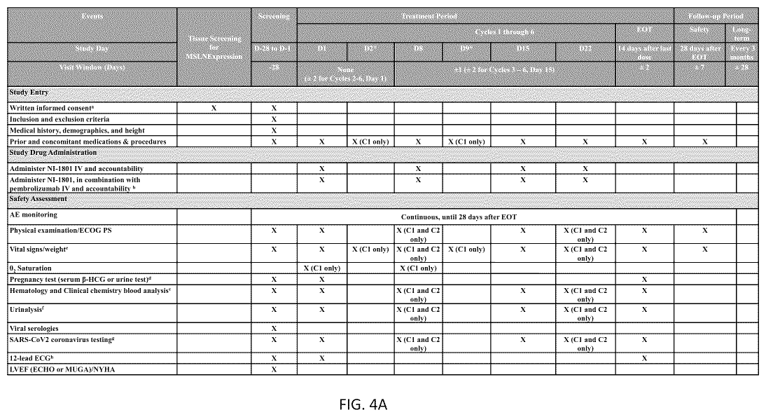

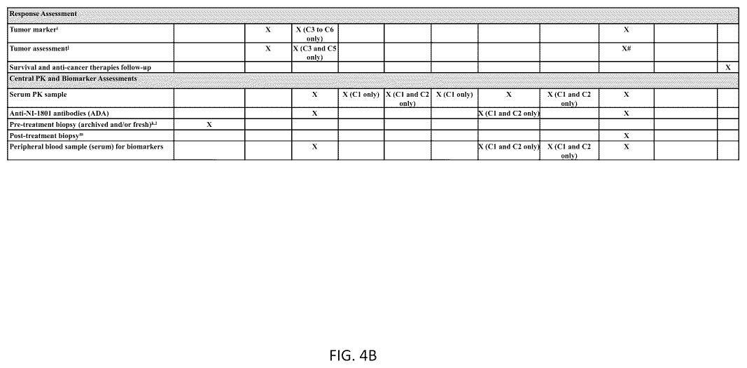

All subjects enrolled in the study described here are closely monitored for safety and tolerability (FIG. 4). Taking into account the measures taken to minimize risk to participants in this study, the potential risks identified in association with NI-1801 are justified by the anticipated benefits that may be afforded to participants who have no other treatment options.

All the above and the available non-clinical data that supports our hypothesis for NI-1801 as single agent and in combination with anti-PD-1 antibody provides enough evidence for a favorable benefit/risk profile in the proposed study population.

### Study Objectives

### Primary Objectives:

To determine the safety and tolerability of NI-1801 as single agent and in combination with pembrolizumab in subjects with advanced, metastatic, or recurrent ovarian cancer (OC), triple negative breast cancer (TNBC) (in case of treatment with NI-1801 alone), ductal pancreatic adenocarcinoma, or non-small cell lung cancer (NSCLC) expressing MSLN.

To define the non-tolerated toxic dose (NTD), maximum tolerated dose (MTD), and/or the recommended phase 2 dose (RP2D) of NI-1801 as single agent and in combination with pembrolizumab in subjects with advanced, metastatic, or recurrent OC, TNBC (in case of treatment with NI-1801 alone), or NSCLC expressing MSLN. The NTD, MTD, and RP2D will be established for both the first and subsequent doses.

### Secondary Objectives

To evaluate the preliminary efficacy of NI-1801 as single agent or in combination with pembrolizumab in MSLN expressing OC, TNBC (in case of treatment with NI-1801 alone), or NSCLC.

To evaluate the progression-free (PFS) and overall survival (OS) rates.

To characterize the PK of NI-1801 when administered as a single agent and in combination with pembrolizumab.

To evaluate a potential relation between NI-1801 serum levels, clinical activity, and tolerability of NI-1801 as single agent and in combination with pembrolizumab.

To determine the presence, frequency, and functional impact of NI-1801 anti-NI-1801 antibodies (ADAs).

### Exploratory Objectives

The exploratory objectives for this trial are:

To assess pre-treatment (archival and/or fresh) and post-treatment biopsies regarding potential changes within the tumor microenvironment (e.g., macrophage and T cells infiltration and phenotyping). To study levels of CD47 and other potential markers using pre-treatment biopsy (archival or fresh). To assess a potential correlation between the expression of MSLN as well as CD47 and the clinical activity of NI-1801. To evaluate serum sMSLN as PD marker of NI-1801 activity. If required to assess preliminary activity, to analyze changes in tumor circulating DNA in peripheral blood.

### Study Endpoints

### Primary Endpoints

The primary endpoints for this trial are:

Adverse events (AEs) according to National Cancer Institute (NCI) common terminology criteria for adverse events (CTCAE) version 5.0 (2).

Dose-limiting toxicity (DLT), non-tolerated dose (NTD), and maximum tolerated dose (MTD) during the DLT window (Part A only).

### Secondary Endpoints

The secondary endpoints for this trial are:

Time to response, duration of response (DOR), disease control rate (DCR), best overall response (BOR), overall response rate (ORR). In Part A, at MTD/recommended dose level only. Progression-free survival (PFS) and overall survival (OS). In Part A, at MTD/recommended dose level only. Concentration versus time measurements for NI-1801 and PK parameters including maximum plasma concentration (Cₘₐₓ), time to maximum concentration (Tₘₐₓ), terminal half-life (T_{1/2}), area under the curve (AUC) including AUC₀₋ₗₐₛₜ, AUC_{inf} as well as AUCₜₐᵤ, clearance (CL), and volume of distribution during the terminal phase (Vz). Relation between Cₘₐₓ and AUC with response variables as defined in secondary endpoint 1.) and primary endpoints 1.) and 2.). Presence and frequency of ADAs to NI-1801 as well as their impact on Cₘₐₓ and AUC and response variables as defined in secondary endpoint 1).

### Exploratory Endpoints

The exploratory endpoints for this trial are:

Evaluate measures of tumor sensitivity/resistance to NI-1801, in relation to e.g., CD47 and MSLN expression levels in tumor tissue, the immune cell infiltration status, as well as NI-1801 tissue penetration. PD markers of NI-1801 biological activity potentially including, but not limited to, levels of serum sMSLN. Tumor circulating DNA in peripheral blood.

### Study Design

### Overall Study Design

Study LCB-1801-001 is an open-label, Phase 1, dose escalation and expansion, FIH clinical study of NI-1801 in patients with advanced, metastatic, or recurrent solid malignancies expressing MSLN. The trial will be conducted in two parts:

Part A: Dose escalation using a modified accelerated titration design and in combination with pembrolizumab.

Part B: Dose expansion to further evaluate the safety and efficacy of NI-1801.

The dose escalation part (Part A) of the study evaluates the safety and tolerability of escalating doses of NI-1801 to determine the maximum tolerated dose (MTD) and non-tolerated toxic dose (NTD) of NI-1801. The expansion part (Part B) further evaluates the safety and efficacy of NI-1801 administered at or below the MTD in up to 10 additional subjects in order to determine the recommended Phase II dose (RP2D). Treatments will be administered in 28-day cycles for up to 12 months until disease progression, unacceptable toxicity, or Investigator/patient decision to withdraw study consent.

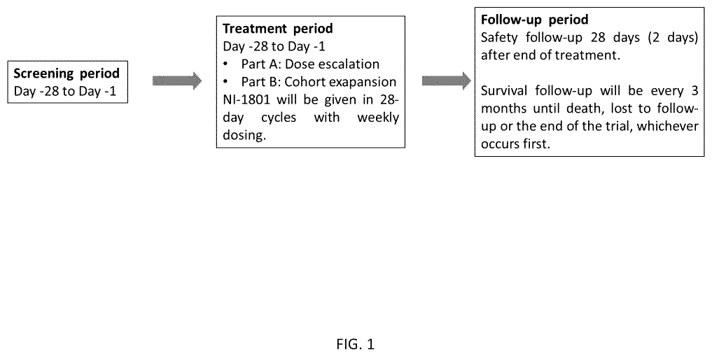

Part A and Part B of the study and sub-study will consist of three phases each with a Screening, Treatment, and Follow-up Period (FIG. 1).

Patients in whom treatment extends beyond 6 cycles will be followed in analogy to procedures and safety follow-up as in Cycle 6 (C6). After discontinuation, the same follow-up as would have been taken place in the trial is offered to the patient.

### Part A Study Design

In the dose escalation phase, and in the combination with pembrolizumab cohort, a modified accelerated titration design (1) establishes initial toxicity of NI-1801 (Error! Reference source not found.). Cohorts of 1 or more subjects will be administered NI-1801 in each dose cohort until ≥1 subject experiences a DLT or ≥2 subjects experience a Grade ≥2 AE, except those toxicities that are clearly and incontrovertibly due to extraneous causes, in the DLT window (Cycle 1, Days 1 to 28). At that time, the current cohort and all subsequent cohorts will be expanded, 3 to 6 subjects per dose level.

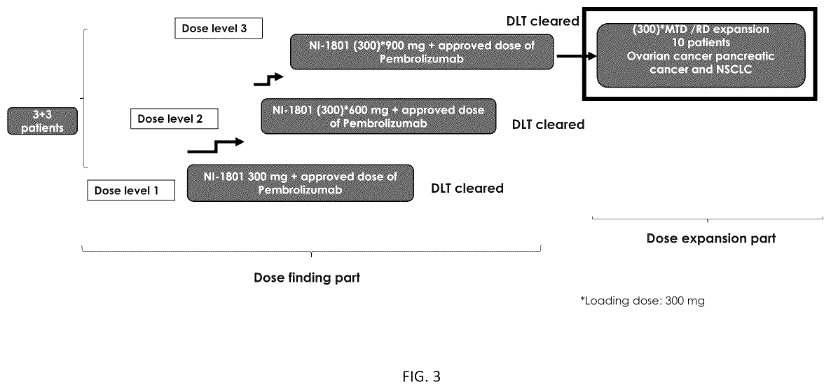

Each subject receives the assigned dose of NI-1801 on Cycle 1, Day 1 (C1D1). The starting dose (Cohort 1) will be 15 mg per subject (fixed dose). Subsequent doses will be given once every two weeks (Q2W), which may be adjusted to every 3 weeks if recommended. All treatments are administered in 28-day cycles; there are no rest periods in-between. In the combination with pembrolizumab cohort, the starting NI-1801 dose is 300 mg (FIG. 3). Pembrolizumab is administered at the dosage of 400 mg every 6 weeks, in 4 cycles. Pembrolizumab is administered as first drug; later, NI-1801 will be infused after 30 minutes.

The initial dose escalation increment in each cohort of the accelerated phase (Cohorts 1 to 3) is 3-fold. The further dose escalation increment will then be ≤2-fold. The number of cohorts depends on the incidence of DLT as well as emergent PK and PD data. Dosing cohorts are summarized as follows:

| | No. of | Cycle 1 Day 1 | Dose increments |
|---|---|---|---|
| Cohort no. | subjects | dose (mg) | (x-fold) |
| 1 | 1 | 15 | |
| 2 | 1 | 50 | 3.3 |
| 3 | 1 | 150 | 3 |
| 4 | 3 | 300 | 2 |
| 4a | 3 | 450 | 1.5 |
| 5 | 3 | 600* | 2 |
| 5a | 3 | 750 | 1.25 |
| 6 | 3 | 900* | 1.5 |
| 7 | 3 | 1200* | 0.75 |

*All patients should receive in C1D1 a starting dose of 300 mg followed by subsequent dose as defined by the SRC in the dose escalation cohorts or the recommended dose in the expansion cohort. The above should apply to the single agent and the combination cohorts. Furthermore, in the combination with pembrolizumab cohort, the starting NI-1801 dose will be 300 mg and 4 dose-levels of NI-1801 are foreseen (until 1200 mg).

In all patients treated in the single agent and in the combination cohorts, at doses equal to or higher than 600 mg, a loading dose of 300 mg is administered on cycle 1, day 1 (C1D1), the actual cohort dose will be administered Q2W starting from C1D8. The intention of this loading dose is to improve the safety and tolerability of NI-1801.

After the first dose is administered in any cohort, subjects in each cohort are observed for at least 28 days (Cycle 1, Days 1 to 28; i.e., dose limiting activity (DLT) window) before the next higher, protocol-specified dose can be administered to any subject. No more than one subject per day will be enrolled in a given dose escalation cohort.

A subject evaluable for DLT is defined as one who:

Has received at least 3 doses of NI-1801 during Cycle 1, Days 1 to 28 (or at least 75% of the total planned Cycle 1 dose intensity) without experiencing a DLT or experienced a DLT after receiving at least one dose or fraction thereof of NI-1801 during Cycle 1, Days 1 to 28.

Subjects non-evaluable for DLT will be replaced.

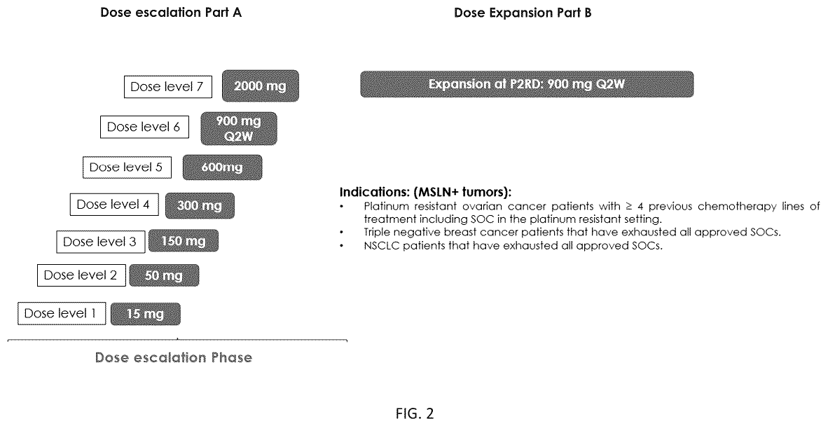

A dose is considered the NTD when 2 or more of up to 6 evaluable subjects in a cohort experience a DLT in the DLT window. The MTD is defined as the last dose below the NTD with 0 or 1 out of 6 evaluable subjects experiencing a DLT during the DLT window. An intermediate dose of NI-1801 (one between the NTD and the last dose level before the NTD) may be evaluated to accurately determine the MTD in Part A. An illustrative example of dose escalation with the resulting hypothetical NTD and MTD is shown in FIG. 2.

Intra-patient dose escalation is permitted (by SRC approval) for patients who completed Cycle 1 without drug-related toxicity of Grade ≥2 and achieved a clinical benefit, i.e., SD or better. Intra-patient dose escalation is allowed once per patient provided that the dose level to which the patient will escalate has already been declared safe, i.e., a DLT rate <33% at the time of the proposed dose increase.

### Part B Study Design

Following completion of dose escalation within Part A of the study, subjects may be enrolled into an expansion phase (Part B) with up to additional 10 subjects (to ensure at least 10 subjects treated at the recommended Phase II dose (R2PD)) to evaluate the RP2D.

Expansion may occur at the MTD established in the dose escalation portion, or at an alternative tolerable dosing schedule, based on review of safety, PK and PD data from Part A by the SRC. In all patients treated in the single agent and in the combination cohorts, at doses equal to or higher than 600 mg, a loading dose of 300 mg will be administered on C1D1, the actual cohort dose will be administered from C1D8. The intention of this loading dose is to improve the safety and tolerability of NI-1801. For Part B, patients may be enrolled simultaneously without an observation time between patients. An illustrative example of dose escalation during Part A with the resulting hypothetical NTD and MTD for cohort expansion in Part B is provided in Error! Reference source not found..

### Inclusion Criteria

Subjects meeting all of the following criteria will be considered for admission to the trial: Adults ≥18 years of age at the time of signing the informed consent form (ICF). Histologically or cytologically confirmed diagnosis of epithelial OC (high-grade serous or endometroid), TNBC, or non-squamous NSCLC. For the combination with pembrolizumab, only subjects with histologically or cytologically confirmed diagnosis of epithelial OC (high-grade serous or endometroid) or non-squamous NSCLC and ductal pancreatic adenocarcinoma. MSLN expression with staining intensity of ≥2+ as per IHC in ≥40% of tumor cells. Staining for MSLN expression can be performed using archival tumor tissue and is foreseen to be performed at the institution's pathology. A slice for centralized IHC assessment for validation and biomarker analysis is mandatory. Patients with advanced, metastatic, or recurrent disease after at least 1 prior systemic treatment for the primary malignancy and who have failed treatment with, are intolerant to, or are not candidates for available therapies that are known to confer a clinical benefit to patients with these tumor entities. Patients treated in either the single agent recommended dose expansion cohort or in the combination with pembrolizumab cohort should have accessible lesions at screening for baseline and on treatment biopsies.

Note: If in a patient treated in any of these two cohorts biopsy is considered by the investigator or and the sponsor's medical monitor to be risky or not feasible, the patient can be treated in any of the above cohorts without performing the study protocol required biopsies.

Eastern Cooperative Oncology Group performance status (ECOG PS) 0-1.

Subjects must have the following laboratory values (determined by local lab):

Absolute neutrophil count (ANC)≥1.0×10⁹/L, the use of colony stimulating factors, i.e., granulocyte colony stimulating factor (G-CSF) or granulocyte-macrophage colony stimulating factor (GM-CSF), within 14 days before the test is not allowed

Platelets ≥100×10⁹/L, transfusion support within 14 days before the test is not allowed Hemoglobin ≥10 g/dL. Prior RBC transfusion is permitted.

Potassium within normal limits or correctable with supplements

Aspartate aminotransferase (AST) and alanine aminotransferase (ALT)≤2.5×upper limit of normal (ULN)

Serum bilirubin <1.5×ULN

Calculated glomerular filtration rate of ≥45 mL/min/1.73 m², according to the MDRD abbreviated formula

International normalized ratio (INR)<1.5×ULN and partial thromboplastin time (PTT)<1.5×ULN

Negative pregnancy test at inclusion (female of childbearing potential [FCBP]).

Females of childbearing potential (FCBP) must: (i) have two negative urine or serum pregnancy tests as verified by the Investigator prior to starting NI-1801; the subject may not receive NI-1801 until the Investigator has verified that the result of the pregnancy test is negative. A urine or serum pregnancy test is required at screening and within 72 hours prior to dosing on Cycle 1, Day 1, and within 72 hours prior to Day 1 of every subsequent cycle. Note: The Cycle 1, Day 1 pregnancy test does not need to be repeated if the screening pregnancy test was done within 72 hours prior to dosing. A serum or urine pregnancy test (Investigator's discretion) must also be performed at the end of study for each FCBP; and (ii) agree to use and be able to comply with a highly effective birth control method, i.e., one that can achieve a failure rate of less than 10% per year when used consistently and correctly (please refer to the Clinical Trials Facilitation and Coordination Group guideline for details), from signing the ICF, throughout the study, and for up to 28 days following the last dose of NI-1801.

Males must be willing to use 1 effective method of contraception during the study, e.g., agree to use a condom (a latex condom is recommended) during sexual contact with a pregnant female or a FCBP, and to avoid conceiving from signing the informed consent form (ICF), while participating in the study, during dose interruptions, and for at least 28 days following NI-1801 discontinuation, even if he has undergone a successful vasectomy.

All patients must agree to abstain from donating blood while taking study medication and for three months following discontinuation of study medication.

Ability of subject to understand character and individual consequences of clinical trial.

The subject is willing and able to adhere to the study visit schedule and other protocol requirements.

Subject must understand and voluntarily sign an ICF prior to any study-related assessments/procedures being conducted.

Life expectancy of at least 2 months.

### Exclusion Criteria

Subjects presenting with any of the following criteria will not be included in the trial:

Patient has known hypersensitivity to NI-1801 or any of the constituent compounds.

Radiotherapy to the target lesions within 4 weeks prior to the first NI-1801 infusion.

Prior anti-cancer therapy including chemotherapy, hormonal therapy, and investigational agents within 2 weeks or within ≤5 half-lives prior to starting NI-1801 dosing (up to a maximum of 4 weeks), whichever is longer. The maximum required washout period will thus not exceed 4 weeks prior to the day of first treatment with NI-1801. Note: Low dose steroids (oral prednisone or equivalent ≤20 mg per day, including systemic or topic use), localized noncentral nervous system (CNS) radiotherapy of non-target lesions, and treatment with bisphosphonates and RANKL inhibitors are not criteria for exclusion.

Other investigational therapies must not be used, i.e., treatment within another clinical trial is not permitted, while the patient is on study. COVID-19 vaccination is allowed only starting from Cycle 2 (if not completed before study inclusion).

Severe cardiac dysfunction (NYHA classification III-IV).

Significant hepatic dysfunction (serum bilirubin ≥1.5 mg/dL or AST and/or ALT≥2.5 times normal level), unless related to liver metastasis.

Patients with known human immunodeficiency virus (HIV) infection or known history or serological evidence of prior hepatitis B or C virus infection. Active SARS-COV2 infection.

Uncontrolled active systemic bacterial, viral, fungal, or other infection (defined as exhibiting ongoing signs/symptoms related to the infection and without improvement, despite appropriate antibiotics or other treatment), or intravenous anti-infective treatment within 2 weeks prior to first dose of NI-1801.

Patients with concomitant active malignancy, requiring ongoing systemic treatment.

Patients with known CNS metastases.

Platelet count <100×10⁹/L (transfusion support within 14 days before the test is not allowed).

Hemoglobin <10.0 g/dL. Prior RBC transfusion is permitted.

ANC <1×10⁹/L (the use of colony stimulating factors, G-CSF or GM-CSF, within 14 days before the test is not allowed).

### Pregnancy and Lactation.

History of psychiatric illness or substance abuse likely to interfere with ability to comply with protocol requirements or give informed consent.

Significant medical diseases or conditions, including laboratory abnormalities, as assessed by the Investigators and Sponsor, which would substantially increase the risk-benefit ratio of participating in the study. This includes, but is not limited to, acute myocardial infarction within the last 6 months, unstable angina, uncontrolled diabetes mellitus, and severely immunocompromised state, major surgery ≤4 weeks prior to starting NI-1801.

Prior treatment with a CD47, SIRPα, or MSLN targeting agent.

Patients in whom acute toxic effects of any prior radiotherapy, chemotherapy, or surgical procedure have not resolved to Grade ≤1 or returned to baseline except for alopecia (any grade), anemia, and peripheral neuropathy (for the latter, recovery to Grade <2 is acceptable).

People who are detained through a court or administrative decision, receiving psychiatric care against their will, adults who are the subject of a legal protection order (under tutorship/curatorship), people who are unable to express their consent, and people who are subject to a legal guardianship order.

Furthermore, subjects presenting with any of the following criteria will not be included in the sub-study (combination with pembrolizumab cohort):
History of/active non-infectious pneumonitis or interstitial lung disease.
Known hypersensitivity to pembrolizumab or excipients.
Participants with an active, known or suspected autoimmune disease. Participants with type I diabetes mellitus, hypothyroidism only requiring hormone replacement, skin disorders not requiring systemic treatment, or conditions not expected to recur in the absence of an external trigger are permitted to enroll.
Prior organ or tissue allograft.
History of Grade ≥3 toxicity related to prior T-cell agonist or checkpoint inhibitor therapy, except those that are unlikely to re-occur with standard countermeasures.
History of myocarditis, regardless of etiology.

### Procedures

The procedures conducted for each patient enrolled in the study are outlined in FIG. 4. All study treatment visits/procedures will have a ±1-day window in Cycles 1 and 2 or ±2-day window in subsequent Cycles unless otherwise specified (e.g., PK sampling) below or in FIG. 4.

All laboratory blood samples should be drawn pre-dose unless otherwise specified (e.g., PK samples). In the single agent dose escalation trial and in the combination with pembrolizumab, taking into account the new modifications, over the course of a full year, with 28 hospital visits scheduled, the estimated maximum volume of blood that may be collected ranges between 765 mL, assuming the upper end of 25-35 mL range per visit.. Specific laboratory tests that must be completed within 28 days prior to the first dose of NI-180. Imaging must be performed within 28 days prior to start of NI-1801 treatment medication.

All study procedures should be recorded in the source document and the electronic case report forms (eCRF). In the event subjects fail screening, minimal information will be documented on the eCRFs, per database instructions.

### Screening Period

### Tissue Screening for MSLN-Expression

A separate signed written informed consent for the tissue type screening for MSLN-expression is obtained before collection and analysis of archival or fresh tumor tissue.

Archival tumor tissue (formalin-fixed, paraffin-embedded (FFPE) slides) or fresh tumor biopsy will be obtained for the analysis of MSLN-expression by standard IHC assay (e.g., using monoclonal antibody clone 5B2 or VENTANA MSLN SP74 assay) at the institution's pathology. A tissue slice for centralized IHC assessment for validation and biomarker analysis is mandatory.

The tumor tissue must be obtained and analyzed before the subjects could be declared eligible, i.e., staining intensity of ≥2+ as per IHC on ≥40% of tumor cells, to sign the written informed consent for the general screening and begin the general screening procedures.

### General Screening

The general screening will only begin after the subject has been declared eligible regarding the tissue analysis for MSLN-expression).

All participants must be provided with an Institutional Review Board/Research Ethics Committee (IRB/REC) approved informed consent document (ICD) describing the study with sufficient information and time for participants to make an informed decision regarding their participation. The ICD will be provided at the Screening visit to all subjects by qualified study staff. It must be signed and dated by the subject and the responsible staff prior to the start of any study procedures and its completion documented in source documents and in the eCRF. The participant must receive a copy of the signed and dated consent documents. A signed copy (in paper or electronic format) of the consent documents must be retained in the medical record or research file.

All patients who enter the general screening period for the study, which starts when the patient signs the ICF, receive a unique subject identification number before any study procedures are performed.

This number is used to identify the patient throughout the clinical trial and must be used on all study documentation related to that patient, including if a patient is rescreened.

### Pharmacodynamic and Biomarker Assessments

Biomarker assessments will be performed centrally and may include, but are not limited to, levels of sMSLN and other soluble factors, and characterization of tumor tissue and the microenvironment.

Sample specimens will be collected as indicated below:

| Biomarker assessment type | Sample type |
|---|---|
| Soluble Mesothelin | Serum |
| Tumor circulating DNA | |
| Target and tumor characterization | Tumor biopsy |
| Immune cell composition | |
| NI-1801 tissue penetration | |

Time points for collection of peripheral blood are outlined below:

| | | Number of | Nominal time points for serum |
|---|---|---|---|
| | Study | serum | sample collection (with allowable |
| | visit | samples to | time window, when applicable) |
| | day | be collected | Serum sMSLN levels |
| Cycle 1-2 | Day 1 | 1 | Pre-infusion (−30 to −5 min) |
| | | | 30 min post-infusion (±10 min) |
| | Day 15 | 1 | Pre-infusion (−30 to −5 min) |
| | | | 30 min post-infusion (±10 min) |
| Cycle 2 | Day 22 | 1 | Pre-infusion (−30 to −5 min) |
| | | | 30 min post-infusion (±10 min) |
| Cycle 3-6 | Day 1 | 1 | Pre-infusion (−30 to −5 min) |
| | | | 30 min post-infusion (±10 min) |
| EOT | 2 weeks | 1 | 2 weeks (±2 days) from last dose |
| | post-dose | | 30 min post-infusion (±10 min) |

### Example 4: Phase I Clinical Update in Patients with MSLN-Expressing Advanced, Metastatic, or Recurrent Cancer

### Single Agent Dose Escalation/Dose Expansion Cohort

With 18^{th} of November as data cutoff, 25 patients enrolled in the dose escalation cohorts at doses: 15 mg, 50 mg, 150 mg, 300 mg, 600 mg, 900 mg and 2000 mg. The first 3 cohorts (15 mg, 50 mg, 150 mg) had one patient each, the 300 mg cohort had 3 patients, 6 patients enrolled at the 600 mg cohort and 12 patients in the 900 mg cohort and 1 patient at 2000 mg.

The Safety Review Committee (SRC), approved in Aug. 28, 2024, 900 mg Q2W as the recommended Phase II dose (RP2D). 12 patients have been enrolled at the RP2D (three of them started at 900 mg once weekly (QW) and then switched to 900 mg Q2W).

No DLTs were reported in these cohorts, NI-1801 was safe and well tolerated, all emerging related adverse events were G1 or G2, with the exception of a transient G3 thrombocytopenia at 900 mg QW, that return to baseline holding the dose two weeks later, the PK of this patient showed accumulation of 30% with the weekly schedule at this dose level and resume treatment with 900 mg every two weeks with stable hematological counts, and 1 patient treated at 2000 mg Q2W experienced transient G3 anemia with G3 transient episode of nausea and vomiting

The above observed accumulation (in this and another two patients at 900 mg QW) triggered the amendment to change the schedule to Q2W.

No anti-tumor activity observed at the first 3 doses levels (15 mg-150 mg QW).

At 300 mg QW, 2 platinum resistant ovarian cancer and 1 triple negative breast cancer (TNBC) patients were enrolled and 1 out of 3 patients enrolled in this cohort (ovarian cancer), experienced disease stabilization (SD) per response evaluation criteria in solid tumors (RECIST) criteria.

At 600 mg QW, 4 platinum resistant ovarian 1 non-small cell lung cancer (NSCLC) and 1 TNBC cancer patients were enrolled, all 4 ovarian cancer patients had SD, 1 of them during almost one year and the other 3 ovarian cancer patients the stable disease (SD) ranged from 4 to 5 months.

At 900 mg Q2W, 12 platinum resistant ovarian cancer patients enrolled (3 of them, started with 900 mg QW and continued after 1 cycle every 2 weeks), 4 patients currently ongoing, 1 patient reached partial response (PR) per RECIST criteria and is ongoing in the trial after 12 months, 1 patients ongoing with stable disease (SD) after 2 months, 1 patient had SD after 2 months and discontinued by investigator decision at week 12, 2 patients ongoing before first tumor assessment.

At 2000 mg Q2W, 1 platinum resistant ovarian cancer patient was enrolled, this patient had transient G3 anemia with an episode of G3 nausea and vomiting that recovered holding the dose two weeks and was discontinued by investigator's decision.

NI-1801 in Combination with Pembrolizumab 400 mg Q6W Dose Escalation/Dose Expansion Cohort

With 18^{th} of November as data cutoff, 21 patients enrolled in the dose escalation cohorts at 300 mg QW, 600 mg QW and Q2W and 900 mg Q2W in combination with pembrolizumab 400 mg once every 6 weeks (Q6W).

No DLTs were reported in these cohorts, this combination was safe and well tolerated, emerging related adverse events were either G1 or G2, with the exception of on patient that has a transient episode of G4 neutropenia and G3 lymphopenia that did not required treatment and another patient with worsening of amylase and lipase from G2 at baseline to G3 on treatment (increases in amylase and lipase have been observed in patients treated with pembrolizumab), this patient was managed with corticosteroids as per pembrolizumab adverse events management godliness and is continuing study treatment.

Anti-tumor activity observed with this combination with 400 mg pembrolizumab Q6W:

At the NI-1801 300 mg cohort: 5 platinum resistant ovarian cancer patients enrolled, 2 currently ongoing beyond 6 months and escalated to 600 mg, 1 with a partial response (PR) and 1 with SD by RECIST criteria, both ongoing after 7 months in the study.

At the NI-1801 600 mg cohort: 7 platinum resistant ovarian cancer patients enrolled, 3 ongoing, 1 with a PR by RECIST criteria beyond 4 months, 2 with SD by RECIST criteria.

At the NI-1801 900 mg cohort: 7 platinum resistant ovarian cancer and 2 pancreatic cancer patients. 1 ovarian cancer with PR at week 8 tumor assessment, 6 ovarian cancer and 1 pancreatic cancer patients ongoing before first tumor assessment.

### PK and ADA

The PK and ADA analysis in patients treated in the single agent cohort and in the combination with pembrolizumab cohort is ongoing. Preliminary data showed a stable PK up 6 months of treatment. Few patients had low titers of ADA that so far did not have a relevant impact on the PK (C. max or AUC). No differences regarding NI-1801 PK profile between patients treated with single agent and patients treated with NI-1801 in combination with pembrolizumab at any dose level.

### SUMMARY

The preliminary analysis of the emerging clinical data from the NI-1801 study single agent cohort and the NI-1801 in combination with pembrolizumab cohort, shows that NI-1801 as single agent at doses from 15 mg weekly to 900 mg every two weeks is safe and well tolerated. And in combination with pembrolizumab (400 mg Q6W) is safe and well tolerated at doses from 300 mg weekly to 900 mg every two weeks. No DLTs were reported at any dose level.

The Safety Review Committee (SRC) approved 900 mg every two weeks as the phase II recommended dose for both, ni-1801 single agent and NI-1801 in combination with pembrolizumab.

Promising preliminary anti-tumor activity of NI-1801 at doses ranging from 300 mg weekly to 900 mg every two weeks as a single agent was observed. The anti-tumor activity was enhanced in the combination with pembrolizumab with an ORR of 30% in the first 10 patients treated with NI-1801 at either 600 mg or 900 mg in combination with pembrolizumab 400 mg every 6 weeks.

Preliminary emerging PK/ADA data from the patients treated with NI-1801 single agent and with the combination with pembrolizumab, showed an stable PK with low titers of ADA in few patients that did not have a relevant impact in either C. max or AUC.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or preventing cancer in a patient in need thereof, comprising administering to the patient a therapeutically effective amount of a bispecific antibody comprising a first arm that comprises a first amino acid sequence that binds CD47 and a second arm that comprises a second amino acid that binds mesothelin (MSLN);
wherein the bispecific antibody comprises a heavy chain comprising a CDRH1 comprising the amino acid sequence of SEQ ID NO: 225, a CDRH2 comprising the amino acid sequence of SEQ ID NO: 226, a CDRH3 comprising the amino acid sequence of SEQ ID NO: 227,
a kappa light chain comprising a CDRL1 comprising the amino acid sequence of SEQ ID NO: 240, a CDRL2 comprising the amino acid sequence of SEQ ID NO: 242, and a CDRL3 comprising the amino acid sequence of SEQ ID NO: 254, and
a lambda light chain comprising a CDRL1 comprising the amino acid sequence of SEQ ID NO: 286, a CDRL2 comprising the amino acid sequence of SEQ ID NO: 292, and a CDRL3 comprising the amino acid sequence of SEQ ID NO: 298; and
wherein the therapeutically effective amount of the bispecific antibody is about 15 mg to about 1200 mg.

2. The method of claim 1, wherein the bispecific antibody comprises a variable heavy chain comprising the amino acid of SEQ ID NO: 114 and a variable kappa light chain comprising the amino acid sequence of SEQ ID NO: 168 and a variable lambda light chain comprising the amino acid sequence of SEQ ID NO: 222.

3. The method of claim 1, wherein the bispecific antibody comprises a heavy chain comprising the amino acid sequence of SEQ ID NO: 2, a kappa light chain comprising the amino acid sequence of SEQ ID NO: 56, and a lambda light chain comprising the amino acid sequence of SEQ ID NO: 108.

4. The method of claim 1, further administering to the patient a therapeutically effective amount of a monoclonal antibody that binds PD-1.

5. The method of claim 4, wherein the monoclonal antibody that binds PD-1 is pembrolizumab.

6. The method of claim 4, wherein the therapeutically effective amount of the monoclonal antibody that binds PD-1 is about 400 mg.

7. The method of claim 1, wherein the bispecific antibody is administered by intravenous injection.

8. The method of claim 4, wherein the monoclonal antibody is administered by intravenous injection.

9. The method of claim 7, wherein the bispecific antibody is administered as a pharmaceutical composition comprising at least one pharmaceutically acceptable carrier.

10. The method of claim 8, wherein the monoclonal antibody is administered as a pharmaceutical composition comprising at least one pharmaceutically acceptable carrier.

11. The method of claim 9, wherein the administration is once weekly or once every two weeks.

12. The method of claim 11, wherein the bispecific antibody is administered once weekly or once every two weeks for at least one cycle, wherein a cycle is 28 days.

13. The method of claim 12, wherein the bispecific is administered once weekly or once every two weeks for at least six cycles.

14. The method of claim 10, further comprising administering the monoclonal antibody every six weeks for at least 4 cycles, wherein a cycle is 28 days.

15. The method of claim 1, wherein the therapeutically effective amount of the bispecific antibody is 300 mg to 2000 mg.

16. The method of claim 15, wherein the therapeutically effective amount of the bispecific antibody is 300 mg.

17. The method of claim 15, wherein the therapeutically effective amount of the bispecific antibody is 600 mg.

18. The method of claim 15, wherein the therapeutically effective amount of the bispecific antibody is 900 mg.

19. The method of claim 1, wherein the cancer is a solid tumor.

20. The method of claim 1, wherein the cancer expresses MSLN.

21. The method of claim 1, wherein the solid tumor is or is derived from ovarian cancer, breast cancer, lung cancer, head and neck cancer, bladder cancer, melanoma, mesothelioma, colorectal cancer, cholangiocarcinoma, pancreatic cancer, leiomyoma, leiomyosarcoma, kidney cancer, glioma, glioblastoma, endometrial cancer, esophageal cancer, biliary gastric cancer, prostate cancer, or combinations thereof.

22. The method of claim 21, wherein the ovarian cancer is epithelial ovarian cancer or endometrioid ovarian cancer, the breast cancer is triple negative breast cancer, the lung cancer is non-small cell lung cancer, and the pancreatic cancer is pancreatic adenocarcinoma.
